SEQUENCE LISTING (12 sequences)

SEQ ID NO: 1 - DNA, 21 bp, artificial
  salmo salar elongation factor 1-alpha
cccctccaggacgtttacaaa

SEQ ID NO: 2 - DNA, 18 bp, artificial
  salmo salar interleukin-1 beta
atgcgtcacattgccaac

SEQ ID NO: 3 - DNA, 21 bp, artificial
  salmo salar lysozyme (lyz)
ctacaatacccaggccaccaa

SEQ ID NO: 4 - DNA, 20 bp, artificial
  salmo salar interferon gamma 1-like
ccgtacaccgattgaggact

SEQ ID NO: 5 - DNA, 20 bp, artificial
  salmo salar complement C3
gaagaaaggtgagccagatg

SEQ ID NO: 6 - DNA, 19 bp, artificial
  salmo salar heat shock cognate 70 kDa protein
cccctgtccctgggtattg

SEQ ID NO: 7 - DNA, 19 bp, artificial
  salmo salar elongation factor 1-alpha
cacacggcccacaggtaca

SEQ ID NO: 8 - DNA, 20 bp, artificial
  salmo salar interleukin-1 beta
ggtccttgtccttgaactcg

SEQ ID NO: 9 - DNA, 21 bp, artificial
  salmo salar lysozyme (lyz)
gtcacaccagtagcggctgtt

SEQ ID NO: 10 - DNA, 18 bp, artificial
  salmo salar interferon gamma 1-like
gggcttgccgtctcttcc

SEQ ID NO: 11 - DNA, 20 bp, artificial
  salmo salar complement C3
tgtgtgtgtcgtcagcttcg

SEQ ID NO: 12 - DNA, 21 bp, artificial
  salmo salar heat shock cognate 70 kDa protein
caccaggctggttgtctgagt

(19) United States
(12) Patent Application Publication
(10) Pub. No.: US 2022/0184159 A1
(43) Pub. Date: Jun. 16, 2022

(54) METHOD OF BOOSTING INNATE IMMUNITY

(71) Applicant: UNIVERSITY OF THE SUNSHINE COAST, Red Hill (AU)

(72) Inventors: Nicholas Andrew PAUL, Red Hill (AU); Alexandra Helene CAMPBELL, Red Hill (AU); Martina JELOCNIK, Red Hill (AU); Michael RIMMER, Red Hill (AU); Valentin THÉPOT, Red Hill (AU)

(21) Appl. No.: 17/416,189

(22) PCT Filed: Dec. 20, 2019
(86) PCT No.: PCT/AU2019/051427

§ 371 Date: Jun. 18, 2021

(30) Foreign Application Priority Data
Dec. 21, 2018 (AU) 2018904916

(51) Int. Cl. A61K 36/04; A61K 35/748; A61K 36/05; A61K 36/03; A61P 37/04

(52) U.S. Cl. CPC A61K 36/04; A61K 35/748; A61P 37/04; A61K 36/03; A61K 36/05

(57) ABSTRACT

The present invention is concerned with boosting innate immunity in animals with an immune system that includes an alternative complement pathway in which they are fed an effective amount of a red seaweed of Asparagopsis species.

### INCORPORATED BY REFERENCE

The Sequence Listing under document MBAPCTUScorrected_ST25.txt, created Feb. 24, 2022 with 3,000 bytes is incorporated by reference.

### TECHNICAL FIELD

The present invention is concerned with boosting innate immunity in animals with an alternative complement pathway.

### BACKGROUND

Any references to methods, apparatus or documents of the prior art are not to be taken as constituting any evidence or admission that they formed, or form, part of the common general knowledge.

Due to the intensification of aquaculture, increased stocking densities, feeding regimes and lower water quality, farmed fish have become more prone to disease outbreaks due to a depressed immune system. Synthetic chemicals and antibiotics have been used to treat and prevent disease outbreaks, but with limited success. Moreover, this strategy brings about a range of detrimental side effects and consequences for the fish, the environment and the end consumers. Ironically, antibiotics can have a negative effect on the immune system of fish. Oxytetracycline (OTC), one of the most commonly used antibiotics, has been shown to have immunosuppressive effects in carp, rainbow trout, turbot and Atlantic cod (Romero, et al., 2012). Lower phagocytic activity, respiratory burst activity and serum immunoglobulin were reported in different fish species exposed to the antibiotics, even at low concentrations. Oxytetracycline is usually administered in the feed (100-150 mg/kg for 10-15 days), and most is ingested by the fish. However, most of the ingested oxytetracycline (70-80%) is excreted intact in the faeces. The effects of antibiotic micropollutants are still largely unknown on wild fish stocks in proximity to aquaculture leases. One known side effect of antibiotic use, other than the direct residues in aquaculture products, is the emergence of antibiotic resistance genes in bacterial populations near aquaculture farms, and this can affect up to 25% of the bacteria present. A study screening the bacterial diversity in ready to eat prawn product found that 80% of the isolated bacteria were resistant to at least one antibiotic. Of these, a few were human pathogens including Escherichia coil, Salmonella, Shigella and Vibrio species (Duran & Marshall, 2005).

Other chemotherapeutics such as hydrogen peroxide, formaldehyde, potassium permanganate and copper sulfate are also commonly used as parasiticides, fungicides and external bactericides. Hydrogen peroxide aside, all these chemicals are cause for concern when they enter the environment with some having reported immunosuppressant activities in fish (e.g. copper sulfate). Another group of chemicals, largely overlooked, whose use is often unrestricted and unregulated, are disinfectants. These chemicals are used to treat aquaculture equipment (e.g. boats and nets) to stop or at least limit the spread of pathogens. They include chlorine-based products (e.g. sodium hypochlorite, chloramine-T and chlorine dioxide), formaldehydes, synthetic phenols and calcium hydroxides (Burridge, et al., 2010). Unfortunately, due to the unrestricted nature of these compounds, little information is available regarding the amount used and the effects these may have, alone or in combination, in areas where their dose could be high (e.g. wharves or small coves). These compounds have known toxic effects on various aquatic organisms. An improvement of the management of fish diseases via better practices (e.g. culture density) and strategies (e.g. immunostimulant feed) could alleviate the need for chemicals to treat and control directly (e.g. antibiotics) or indirectly (e.g. disinfectant) fish disease outbreaks.

The antibiotic issue is the same on land with agriculture (e.g. poultry and pigs) except that the use of antibiotics is higher and for growth promotion practices as well as to treat or prevent diseases (Chang, et al., 2015). The constant use of sub-therapeutic antibiotics is the perfect situation for antibiotic resistant genes to evolve. Because 70% of the antibiotics used in agriculture are also used in human medicine this has the potential to cause severe complications for patients with an antibiotic resistant bacterial infection. Antibiotic resistance in the U.S. alone is costing the health care sector $21 to $30 billion annually, and this is forecast to increase together with the forecasted use of sub-therapeutic antibiotics in agriculture. As for aquaculture, significant efforts have been allocated to finding alternatives to antibiotics, with immunostimulant compounds being one of the most promising avenues.

Immunostimulants have the capacity to enhance the innate immune system, and thus resistance to disease, but also to reduce the impact of stress and sub-optimal farming conditions (e.g. crowding and grading). In addition, some immunostimulants have been shown to reduce the faecal shedding of certain bacteria including human pathogens such as Salmonella species in pigs and in poultry, thus reducing the risk of meat or egg contamination with the pathogenic bacteria (Bouwhuis, et al., 2017). immunostimulants have also been associated with increased intestinal health leading to increased nutrient absorption and better feed efficiencies in both land and aquatic animals (Sohn, et al., 2000; Nawaz, et al., 2018). The supplementation of aquafeed with immunostimulants leads to significant reductions in the rate of infection and mortalities in different fish species exposed to the main aquaculture pathogens including Aeromona salmonicida, A. hydrophila, Vibrio angillarum, V. alginolyticus, V. harveyi, V. parahaemolyticus, Edwardsiella tarda, Yersinia rukeri, Streptococcus iniae, S. agalactiae, Flavobacterium columnare, infectious pancreatic necrosis virus, irridovirus, Saprolognia sp., and parasites such as nematodes, monogeneans and amoeba (Vallejos-Vidal, et al., 2016; Pahor-Filho, et al., 2017; Nawaz, et al., 2018).

There remains a need to effectively boost innate immunity in an animal to reduce the rate of infection and mortalities when the animal is exposed to pathogens and/or to reduce antibiotic and other chemotherapeutic use in animals.

### SUMMARY OF INVENTION

In an aspect, the invention provides a method for boosting innate immunity in an animal with an alternative complement pathway, comprising the step of administering to said animal an effective amount of a red seaweed of Asparagopsis species, an extract therefrom or residual biomass following an extraction process.

In another aspect, the invention provides the use of a red seaweed of Asparagopsis species, an extract therefrom or residual biomass following an extraction process, for boosting innate immunity in an animal with an alternative complement pathway.

In another aspect, the invention provides the use of a red seaweed of Asparagopsis species, an extract therefrom or residual biomass following an extraction process, in the manufacture of a medicament for boosting innate immunity in an animal with an alternative complement pathway.

In another aspect, the invention provides a method of treating or preventing disease in an animal with an alternative complement pathway, comprising the step of administering to said animal an effective amount of a red seaweed of Asparagopsis species, an extract therefrom or residual biomass following an extraction process.

In another aspect, the invention provides the use of a red seaweed of Asparagopsis species, an extract therefrom or residual biomass following an extraction process, for the treatment or prevention of disease in an animal.

In another aspect, the invention provides the use of a red seaweed of Asparagopsis species, an extract therefrom or residual biomass following an extraction process, in the manufacture of a medicament for the treatment or prevention of disease in an animal.

### BRIEF DESCRIPTION OF THE DRAWINGS

Preferred features, embodiments and variations of the invention may be discerned from the following Detailed Description, which provides sufficient information for those skilled in the art to perform the invention. The Detailed Description is not to be regarded as limiting the scope of the preceding Summary of the Invention in any way.

The Detailed Description will make reference to a number of drawings as follows:

FIG. 1 is a graph showing ACH₅₀ (haemolytic activity) in rabbitfish of various seaweeds and other functional ingredients—Asparagopsis treatment is to the far right;

FIG. 2 is a graph showing phagocytic activity in rabbitfish of various seaweeds—Asparagopsis treatment is to the far right: (a) shows the measurement when the proportion of phagocyte cells containing engulfed beads is compared between treatments (b) shows the measurement when the number of beads per phagocyte cell (phagocytic index) is compared between treatments;

FIG. 3 is a graph showing lysozyme activity in rabbitfish of various seaweeds—Asparagopsis treatment is to the far right;

FIG. 4 is a graph showing respiratory burst in rabbitfish for various seaweeds—Asparagopsis treatment is to the far right;

FIG. 5 is a graph showing ACH50 (haemolytic activity) in salmon of whole Asparagopsis treatment and two doses of a methanolic extract (equivalent to 3% and 6% whole inclusion)—a positive control (Lipopolysaccharide-LPS) is to the far right;

FIG. 6 is a graph showing phagocytic activity of whole Asparagopsis treatment and two doses of a methanolic extract (equivalent to 3% and 6% whole inclusion)—a positive control (Lipopolysaccharide-LPS) is to the far right;

FIG. 7 is a graph showing lysozyme activity of whole Asparagopsis treatment and two doses of a methanolic extract (equivalent to 3% and 6% whole inclusion)—a positive control (Lipopolysaccharide-LPS) is to the far right;

FIG. 8 is a graph showing respiratory burst of whole Asparagopsis treatment and two doses of a methanolic extract (equivalent to 3% and 6% whole inclusion)—a positive control (Lipopolysaccharide-LPS) is to the far right;

FIG. 9 is a graph showing interferon-λ (IFN) gene expression of whole Asparagopsis treatment and two doses of its extract (equivalent to 3% and 6% whole inclusion)—a positive control (Lipopolysaccharide-LPS) is to the far right: (a) shows the measurement of gene expression relative to control (0 value) when a tissue sample from the liver is taken (b) shows the measurement of gene expression relative to control (0 value) when a tissue sample from the kidney is taken;

FIG. 10 is a graph showing interieukine-1β gene expression of whole Asparagopsis treatment and two doses of its extract (equivalent to 3% and 6% whole inclusion)—a positive control (Lipopolysaccharide-LPS) is to the far right: (a) shows the measurement of gene expression relative to control (0 value) when a tissue sample from the liver is taken (b) shows the measurement of gene expression relative to control (0 value) when a tissue sample from the kidney is taken;

FIG. 11 is a graph showing heat shock protein-70 gene expression of whole Asparagopsis treatment and two doses of its extract (equivalent to 3% and 6% whole inclusion)—a positive control (Lipopolysaccharide-LPS) is to the far right: (a) shows the measurement of gene expression relative to control (0 value) when a tissue sample from the liver is taken (b) shows the measurement of gene expression relative to control (0 value) when a tissue sample from the kidney is taken;

FIG. 12 is a graph showing complement protein-3 gene expression of whole Asparagopsis treatment and two doses of its extract (equivalent to 3% and 6% whole inclusion)—a positive control (Lipopolysaccharide-LPS) is to the far right: (a) shows the measurement of gene expression relative to control (0 value) when a tissue sample from the liver is taken (b) shows the measurement of gene expression relative to control (0 value) when a tissue sample from the kidney is taken;

FIG. 13 is a graph showing lysozyme-C gene expression of whole Asparagopsis treatment and two doses of its extract (equivalent to 3% and 6% whole inclusion)—a positive control (Lipopolysaccharide-LPS) is to the far right: (a) shows the measurement of gene expression relative to control (0 value) when a tissue sample from the liver is taken (b) shows the measurement of gene expression relative to control (0 value) when a tissue sample from the kidney is taken;

### DETAILED DESCRIPTION OF PREFERRED EMBODIMENTS

Asparagopsis is a genus of red algae. The genus presently comprises two species, Asparagopsis armata and Asparagopsis taxiformis. Asparagopsis species are marine algae ('seaweeds'). Asparagopsis armata is native to the Southern Hemisphere, having first been described in Western Australia in 1855. According to AlgaeBase (www.algaebase.org) Asparagopsis armata has been introduced into the Mediterranean Sea, and is now frequent and widespread in the western Mediterranean. Like many red algae, its life cycle has two distinct phases that are very different in appearance (although the biochemistry is remarkably similar between the two phases e.g. Paul, et al., 2006; Verges, et al., 2008); the gametophyte phase and the tetrasporophyte phase. The gametophyte phase is most abundant in Australia and Europe between June and December. It is pale purple-red, and it has irregularly branched thalli that are typically 20 mm wide and up to 200 mm long. The lower branchlets of Asparagopsis armata have characteristic harpoon-like barbs, leading to the common name of “harpoon weed”. The tetrasporophyte phase (previously identified as Faikenbergia rufolanosa) occurs in Australia and Europe all year round. It is brownish red, branched and filamentous and grows in 1 mm diameter tufts.

Asparagopsis taxiformis is distributed in tropical/subtropical oceans from Rottnest Island, Western. Australia to southern Queensland. A. taxiformis does not have barbs. In Australia it is commonly referred to as “iodine” weed as it smells like an iodine tincture. The species has been introduced to the Mediterranean Sea by shipping. Asparagopsis taxiformis also has a haplodiplophasic lifecycle, with the haploid phase previously being identified as Falkenbergia hillebrandii.

For the avoidance of doubt, references to “Asparagopsis” generally, “Asparagopsis species”, “Asparagopsis app.” or “Asparagopsis sp.” refers to all species in the genus Asparagopsis. Since taxonomic names can change, and species can be re-classified, the term also refers to species within the genus named using previous names and species within any future genus covering the organisms presently in the genus Asparagopsis.

The present invention relates to the finding that red seaweed of Asparagopsis species is a novel and effective immunostimulant for boosting the innate immune system in animals with an immune system that includes an alternative complement pathway. Therefore, treatment of animals such as fish with red seaweed of Asparagopsis species is an alternative to other disease management strategies such as use of antibiotics, either continuously or in response to infection, vaccines and chemotherapeutics such as, for fish, bathing in hydrogen peroxide.

For the avoidance of doubt, in the context of the present invention, the term “treatment” includes references to therapeutic treatment of animals in need of such treatment, as well as to the prophylactic treatment of animals which are susceptible to the relevant disease states.

The person skilled in the art will appreciate that prophylaxis or treatment of animals such as fish with red seaweed of Asparagopsis species may also be used in conjunction with other disease management strategies.

In an embodiment, administration of the red seaweed complements other options for managing disease in animals. For example, administration of the red seaweed may reduce the necessity for vaccination or other interventions such as chemical bathing to clean fish or freshwater bathing of saltwater fishes (which is currently a treatment for amoebic gill disease). Vaccination may be, for example, against pilchard orthomyxovirus. Stimulation of the innate immune system would make the fish more resistant to secondary infection, which is advantageous if amoebic gill disease and if stress (for example, higher temperature) become more frequent. The red seaweed may also be administered in conjunction with antibiotic use, be that regular antibiotic use for prophylactic effect or administration of antibiotics when a population is threatened by or subjected to disease. It is likely that other interventions can be reduced or lessened in frequency when used in conjunction with administration of the red seaweed of the present invention. Regular antibiotic doses for prophylactic effect can be reduced or discontinued, and the need to administer treatments such as antibiotics when animals are in a diseased state is likely to be reduced.

The administration of the red seaweed leads to significant reductions in the rate of infection and mortalities in animal species exposed to pathogens. The red seaweed may be administered either in response to an immune challenge when infection is present or to prevent infection. By way of example, a reduction in infection rates and mortalities may be expected in fish species exposed to the main aquaculture pathogens including Aeromona salmonicida, A. hydrophila, Vibrio angillarum, V. alginolyticus, V. harveyi, V. parahaemolyticus, V. salmonicida, Edwardsiella tarda, Pseudomonas fluorescens, Ichthyophtirius multifillis, Yersinia rukeri, Streptococcus iniae, S. agalactiae, Flavobacterium columnare, Renibacterium salmoninarum, Mycobacterium species, Photobacterium damselae, Chlamydia-like bacteria, infectious pancreatic necrosis virus, infectious haematopoietic necrosis virus, infectious salmon anemia virus, haemorrhagic septicaemia virus, aquabirnaviruses, irridovirus, nodovirus, betanodavirus, herpervirus (including pilchard herpesvirus or orthomyxovirus and cyprinid herpesvirus), Saprolegnia species, and parasites such as Amyloodinium ocelatum, Cryptocaryon irritans, Ichthyophonus hoferi, copepods, isopods, Trichodina species, nematodes, monogeneans, myxozoans and amoeba. Likewise, a reduction in infection rates and mortalities may be expected in animal species exposed to the pathogens or conditions including bovine viral diarrhoea, bovine respiratory syncytial virus, parainfluenza, proliferative enteropathy, sarcoptic mange, farrowing sickness, infectious bovine rhinotracheitis, Haemophilus sommus, clostridial disease, pleuropneumonia, exudative dermatitis, swine dysentery, mastitis, parvovirus, Campylobacter species, infectious coryza, pasteurellosis, coccidiosis, bordetellosis, tuberculosis, salmonellosis (e.g. pullorum disease), campylobacteriosis, colibacillosis, aspergilosis, listeriosis, leptospirosis, chytridiomycosis, zygomycoses, chromomycoses, ichthyopphoniasis, saprolegniasis, laryngotracheitis, mycoplasmosis, mycobacteriosis, staphylococcosis, trichinellosis, streptococcosis, dermatophilosis, chlamydiosis, adenoviral hepatitis, adenovirus, coronavirus, haemorrhagic enteritis, parvovirusinfectious bronchitis, cystitis, nonspecific septicaemia, erysipelas, influenza (including canine, avian, feline, swine, equine, bovine), fading chick syndrome, fowl pox, crocodile pox, caiman pox, winter sores, kennel cough, feline immunodeficiency virus, feline leukemia virus, feline calcivirus, herpesvirus, distemper, Newcastle disease, enteritis, libyostrongylosis, cholera, Marek's disease, sheep retrovirus, enterotoxemia, sheep pox, Q fever, rabies, ranavirus, anthrax, rinderpest and eczema.

In an embodiment the red seaweed is Asparagopsis taxiformis.

In an embodiment the red seaweed is Asparagopsis armata.

As would be well understood by the person skilled in the art, the innate immune system is one of the two main immunity strategies found in vertebrates (the other being the adaptive immune system). There are a number of components to the innate immune system. Major functions include recruitment of immune cells to sites of infection through the production of chemical factors such as cytokines, activation of the complement system and activation of leukocytes. Three biochemical pathways activate the complement system: the classical complement pathway, the alternative complement pathway, and the lectin pathway. Over 30 proteins and protein fragments make up the complement system, including serum proteins, and cell membrane receptors. The end result of complement activation is stimulation of phagocytes to clear foreign and damaged material, inflammation to attract additional phagocytes, and activation of the cell-killing membrane attack complex.

The classical pathway is triggered by activation of the C1-complex. The C-1 complex comprises the C1q, C1r and C1 s proteins. Activation occurs when C1q binds to IgM or IgG complexed with antigens or if it binds directly to the surface of the pathogen. The lectin pathway is homologous to the classical pathway, but with the opsonin, mannose-binding lectin (MBL), and ficolins, instead of C1q. The lectin pathway is activated by a range of microbes presenting certain types of sugar moieties (e.g. mannans or N-acetylglucosamine).

The alternative pathway is continually active at a low level. This pathway can be activated by a range of microorganisms, including bacteria, viruses, fungi and protozoans. C3 protein hydrolyses spontaneously in the cell environment but the hydrolysis products are immediately inactivated. However, when C3 reacts with a hydroxyl or amino group of a molecule on the surface of a pathogen, the C3b produced is covalently bound to the surface and so is protected from inactivation. This creates a cascade in which further C3 protein is cleaved and becomes bound to the surface of the pathogen to ultimately establish a membrane attack complex. The membrane-attack complex (MAC) forms transmembrane channels. These channels disrupt the cell membrane of target cells, leading to cell lysis and death.

The extent of activation of the alternative complement pathway can be established by measuring the haemolytic activity of a serum sample. The alternative complement pathway haemolytic activity (ACH) is generally reported as the reciprocal of the serum dilution causing 50% lysis of red blood cells (ACH₅₀) and quantifies the haemolytic activity of a serum sample (units/ml). The ACH₅₀ value correlates to the extent of activation of the alternative pathway (Montero, et al., 1998).

Administration of red seaweed of Asparagopsis species involves a key immune parameter, ACH₅₀ (serum haemolytic activity). Accordingly, in an embodiment the invention provides a method which involves feeding red seaweed of Asparagopsis species to a fish to increase haemolytic activity.

Administration of red seaweed of Asparagopsis species also leads to an increase in phagocytic activity. Additionally, administration of red seaweed of Asparagopsis species does not negatively impact on other aspects of immunology in fish.

As used herein in relation to boosting the innate immune system in animals with an immune system that includes an alternative complement pathway, an “effective amount” of a seaweed is an amount that provides a significant increase in activation of the alternative complement pathway. An increase in activation of the alternative complement pathway can be measured by establishing an increase in the haemolytic activity of a serum sample from fish fed the seaweed compared to a serum sample from fish not fed the seaweed. For example, a fish feed supplemented with an effective amount of red seaweed can be compared with the same fish feed lacking the supplement (as in Example 1 below, where the fish fed conventional fish feed act as a control). Preferably the fish feed provides a balanced diet and can be manufactured at acceptable cost, stored for extended periods of time without deterioration, and easily packaged and handled, such as a commercial fish feed formulation supplemented with an effective amount of red seaweed.

As used herein, a “significant” improvement in activation of the alternative complement pathway is defined as a statistically significant improvement in activation as measured by any reliable means which would be employed by the person skilled in the art for measuring the such activation. In particular, such an improvement will be measured by establishing a statistically significant increase in the haemolytic activity of a serum sample from fish fed the red seaweed compared to a serum sample from fish not fed the seaweed, preferably an improvement of at least 5% or more, more preferably at least 10-25%, more preferably at least 100%, more preferably at least 150% or more.

Among jawed vertebrates (teleosts, reptiles, birds, amphibians and mammals), the humoral components of the innate immune system including the complement system, lysozyme, antimicrobial peptides and natural antibodies are the most conserved, showing the same basic functions and sharing their main properties. In jawed vertebrates, the complement system can be activated in three ways, the classic pathway (e.g. IgM in humans and IgG in fish), the alternative pathway (spontaneous activation of C3 by close proximity to antigen) and the lectin pathway (recognition of microbes' surface). The end results of the activation of any of the three pathways are cytolysis of pathogens, phagocytosis, inflammation and solubilisation of immune complexes (Boshra et al., 2006). The mammalian and teleost complement system have been extensively investigated. In mammals, nine key proteins have been identified: C1, C2, C3, C4, C5, C6, C7, C8 and C9 with Factor I as the decay accelerating factor (C3b and C4b proteolysis) and Factor H as the enzymatic inhibitor of C3 convertase activity. In comparison, in fish the proteins identified in the complement system are: C1, C3, C4, C5, C7, C8 and C9 with Factor I and H-like activities shown in various teleosts (Riera Roma et al., 2016). Compared to mammals and other jawed vertebrates, teleosts can possess multiple homologues of complement proteins, with four C3 isoforms found in trout. This evolution of multiple C3 molecules may aid in the recognition of diverse pathogenic organisms (Plouffe et al., 2005). Although, some differences exist between the molecules involved in the different pathway, the activation routes and the end results remain the same between fish and mammals. For the avoidance of doubt, the present invention is applicable to any animal with an alternative complement pathway. The terms “animal”, “animals” as used herein include references to mammalian (e.g. human) patients.

In an embodiment, the animal is a non-human animal.

In an embodiment the animal is a jawed vertebrate. The jawed vertebrates include cartilaginous fish, bony fish, birds, mammals, reptiles, including crocodiles and alligators, and amphibians.

In an embodiment the jawed vertebrate is a bird. In an embodiment the bird is one of the poultry species. Poultry species are domesticated birds kept by humans for their eggs, their meat or their feathers. These birds are most typically members of the superorder Galloanserae, especially the order Galliformes. Poultry birds include chickens (including bantams), quails, turkeys, emus, fowls such peafowl and guinea fowl, swans, turkey, geese and ducks.

In an embodiment the animal is a monogastric animal.

In an embodiment the animal is a monogastric animal selected from the group consisting of rats, dogs, pigs, cats, horses and rabbits.

In an embodiment the animal is a ruminant animal.

In an embodiment the ruminant animal is selected from the group consisting of cattle, sheep, bison, buffalo, deer, antelopes, giraffes, camels, and chevrotains.

In an embodiment the animal is one of the bony fishes, particularly one of the teleosts, or ray-finned fishes.

The present invention can be practiced with any of the considerable variety of fresh, brackish, or salt water fish species including, but not limited to: barramundi, catfish, carp, trout, salmon, tuna, cobia, char, whitefish, sturgeon, tench, roach, pike, pike-perch, sole, turbot, halibut, yellowtail, bass, bream, kingfish, milkfish, tilapia, mullet, grouper, eels and aquarium fish such as goldfish, angel fish, clown fish, cichlids, corydoras, danio, discus, eel, gourami, guppy, loach, minnow, molly, platy, Plecostumas, rainbow and platy variatus, rasbora, shark, sword, tetra, botia, knife fish, lionfish, archer fish, flounder, goby, half beak, mono, needle fish, pipe fish, puffer, scat (green and red), rabbitfish, bumble bee, twin spot damsel, yellowtail damsel, barbed squirrel, wrasse, black-spotted puffer, trigger fish, puffer, and butterfly fish. Yet other species with which the present invention can be practiced will be apparent to the person skilled in the art. The person skilled in the art will appreciate that commercial species in a particular country may be determined by availability locally, arid also that they may be named differently in different locations.

In an embodiment the fish is a commercial species that is farmed. In particular, the fish is selected from the group consisting of salmon, tuna, trout, sea bass, turbot, halibut, sea bream, kingfish, barramundi, grouper, carp, tilapia, and catfish. In an embodiment the fish is salmon.

The red seaweed may be administered to the animal by any suitable method. The red seaweed can be administered in a solid form. This may be in the form of dried seaweed, which may be pulverized or powdered. The dried seaweed can be formulated as a veterinary or pharmaceutical composition or as a feed supplement. In an embodiment it is physically mixed with feed material in a dry form. The red seaweed may also be formed into a liquid formulation and thereafter sprayed onto feed material. The red seaweed may be introduced to an animal in any suitable way. For example, it may be spread upon the water for fish to consume or added to drinking water or feed for a land animal.

Animals, including fish, will not generally eat the seaweed in its natural form as they do not find it palatable. Therefore, while the red seaweed may be fed directly to animals, it is preferable to formulate it into an animal feed to make it palatable. In embodiments where the red seaweed is fed directly to animals it would generally be collected and dried before doing so. Advantageously, it would be collected, dried and powdered before feeding to the animal. Typically, it would be collected, dried, powdered and pelletised before feeding to the fish. In an embodiment, the red seaweed would be pelletised along with other animal feed components. In an alternative embodiment, seaweed in powdered or pelletised form is used to supplement animal feed. A supplement may be mixed into an animal feed, administered separately at the same time as the animal feed, or administered at a different time to the animal feed provided that the animal will consume it.

The seaweed may be treated after harvest, for example, to concentrate the bioactives and/or to facilitate storage and/or to facilitate formulation. It can be washed after collection. Washing the seaweed will remove salt, sand and biological contaminants. Advantageously, seaweed is spun to get rid of excess water after washing. In embodiments, parts of the seaweed are used. For example, after harvesting, the upright portions may be cut off, leaving the rhizome/root-like structures. Chemical compounds may be extracted from the seaweed after collection and the compounds, alone or in admixture, may be administered to an animal. Alternatively, the biomass remaining after extraction with a solvent can be administered to an animal. The seaweed, extract or extracted biomass can be dried and administered as a powder, or the powder may be incorporated into a pellet as discussed above. In an embodiment, the seaweed is dehydrated or dried for storage. The seaweed may be dried in various ways including air drying, oven drying and freeze drying.

Methods of extraction of chemical compounds from algae are well understood by the person skilled in the art. By way of example, solvent extraction may be employed. However, other techniques such as super-critical fluid extraction, in which the temperature and pressure of a fluid are raised above their critical point to give characteristics of both liquids and gases, may be used. The extraction may be assisted by exposing the material to high-pressure steam and/or a water-based solution containing water and/or other suitable solvents. The extraction process can also be effectively assisted by the application of a static and/or an alternating physical field such as heat energy and/or high-frequency alternating physical field, examples of which include but are not limited to: microwave, radio-frequency or an ultrasonic fields. By way of example, extraction may be assisted by the use of techniques such as the application of ultrasound waves with a frequency above 20 kHz to 100 kHz to break down the material. These waves cause the creation of bubbles and zones of high and low pressure. When bubbles collapse in the strong ultrasound field cavitation occurs. The implosive collapse, cavitation, near liquid-solid interfaces causes breakdown of particles, which means that mass transfer is increased and bioactive compounds are released from a biological matrix.

The extraction process typically comprises extraction with an organic solvent or water-based medium. The extraction process may involve, for example, an aqueous alkali-based leaching, but water or an organic solvent may be used. Mixtures of the treatment agents may be used if desired. The extraction may be at an elevated temperature. Typically an extraction is conducted at a temperature from 30° C. to 80° C. Typically the extraction time is from 24 hours to 72 hours.

Compounds may be extracted from the red seaweed with a polar solvent such as water, or a polar organic solvent such as an alcohol, in particular methanol, ethanol, propanol, butanol or hexanol, acetone, ethyl acetate, dimethylsulfoxide, dimethylformamide and tetrahydrofuran. In this case relatively more polar molecules will be extracted. The residue will contain relatively less polar compounds and the balance of the polar compounds not extracted under the extraction conditions used. Compounds that are active in inducing the effects described herein may be extracted but, equally, they could remain in the residue. In the former case it may be expected that the extract will have utility in the present invention, while in the latter it may be the residue that is effective. The skilled person will be able to ascertain with only routine experimentation which solvents are effective in producing an extract that is effective.

Alternatively, compounds may be extracted from the red seaweed with a non-polar solvent such as n-hexane, cyclohexane, benzene, toluene, chloroform, carbon tetrachloride or an ether such as diethyl ether. In this case relatively less polar molecules will be extracted The residue will contain relatively more polar compounds and the balance of the non-polar compounds not extracted under the extraction conditions used. As for polar solvents, the skilled person will be able to ascertain with only routine experimentation which solvents are effective in producing an extract that is effective.

Any reference to “seaweed”, “red seaweed”, “seaweed of Asparagopsis species” or similar in the context of this invention shall, unless the context requires otherwise, be taken to refer to the seaweed itself in any physical form as well as to extracts of the seaweed or the biomass remaining once the seaweed has been subjected to an extraction process.

In an embodiment an extract of the seaweed with a polar solvent is used.

In an embodiment the extract of the seaweed is an extract with a polar solvent selected from the group consisting of water, an alcohol, acetone, ethyl acetate, dimethylsulfoxide, dimethylformamide and tetrahydrofuran.

In an embodiment the polar solvent is an alcohol, in particular methanol, ethanol, propanol, butanol or hexanol, and typically methanol.

In an embodiment the red seaweed, or an extract therefrom or the residue of the seaweed once extracted, is incorporated into an animal feed.

When used in combination with a feed material for land animals, particularly livestock, the feed material is preferably grain/hay/silage/grass-based. Included amongst such feed materials are improved and/or tropical grass or legume based forages, any feed ingredients and food or feed industry by-products as well as bio-fuel industry by-products and corn meal and mixtures thereof, or feed lot and dairy rations, such as those high in grain content. The feed is supplemented with the red seaweed of the invention. Thus, the animal, when feeding, ingests the red seaweed which can then act to boost its innate immune system. In practice, livestock are typically fed the animal feed supplement by adding it directly to the animal feed, e.g. as a so-called top-dress, but it may be incorporated in a compounded animal feeds or used in the preparation or manufacture of lick blocks. A lick block typically comprises various types of binders, e.g. cements, gypsum, lime, calcium phosphate, carbonate, and/or gelatin.

In an embodiment, the red seaweed, or an extract therefrom or the residue of the seaweed once extracted, is incorporated into fish feed which, in addition to the red seaweed, extract or residue, comprises one or more water soluble and/or dispersible nutritional ingredients. Typically, fish feed is in the form of a pellet or crumble which comprises one or more water soluble and/or dispersible nutritional ingredients and other ingredients. Typically the water soluble and/or dispersible nutritional ingredients are vegetable matter, e.g., flour, meal, starch or cracked grain produced from a crop vegetable such as wheat, alfalfa, corn, oats, potato, rice, and soybeans; cellulose in a form that may be obtained from wood pulp, grasses, plant leaves, and waste vegetable matter such as rice or soy bean hulls, or corn cobs; animal matter, e.g., fish and shellfish (e.g., shrimp or crab) meal, oil, protein or solubles and extracts, krill, meat meal, bone meal, feather meal, blood meal, or cracklings. Typically, a fish feed pellet further comprises ingredients such as binders, fillers, vitamins and minerals, amino acids, colourants, chelating agents and stabilisers. In addition, fish feed pellets can comprise antibiotics and other medicinal compounds.

In an embodiment fish feed pellets comprise red seaweed which has been comminuted.

In an embodiment fish feed pellets comprise red seaweed which has been dried and powdered. The powdered red seaweed can be sieved to in order to select seaweed particles of a particular size. In an embodiment, the powdered seaweed is reduced to a particle size of from 10 to 1000 microns. In an embodiment, the powdered seaweed is reduced to a particle size of from 100 to 500 microns. In an embodiment, the powdered seaweed is reduced to a particle size of about 400 microns. Seaweed powder can be incorporated into fish feed pellets.

In an embodiment the fish feed comprises red seaweed, an extract therefrom or residual biomass following an extraction process, in amount from 0.01% w/w to 10% w/w. In an embodiment the fish feed comprises red seaweed in an amount of from 0.5% w/w to 5% w/w. In an embodiment the fish feed comprises whole red seaweed in an amount of from 1% w/w to 3% w/w. Typically the solvent extract has greater effect. In an embodiment, the whole seaweed is administered at 3% w/w while a 3% solvent extract is administered at 0.6% w/w. In an embodiment the fish feed comprises whole red seaweed in an amount of from 1% w/w to 3% w/w. In an embodiment the fish feed comprises an extract from the red seaweed. In an embodiment the extract is administered at 0.01% w/w to 1.5% w/w. In an embodiment the extract is administered at 0.5% w/w to 1.25% w/w.

It is preferable that fish be fed the red seaweed as a component of fish feed pellets, crumbles, or other fish feed forms, e.g., commercially available fish feed, or as an ingredient in a fish feed comprising other well-known ingredients included in commercial fish feed formulations so as to provide a nutritionally balanced complete fish feed, including, but not limited to: vegetable matter, e.g., flour, meal, starch or cracked grain produced from a crop vegetable such as wheat, alfalfa, corn, oats, potato, rice, and soybeans; cellulose in a form that may be obtained from wood pulp, grasses, plant leaves, and waste vegetable matter such as rice or soy bean hulls, or corn cobs; animal matter, e.g., fish and shellfish (e.g., shrimp or crab) meal, oil, protein or solubles and extracts, krill, meat meal, bone meal, feather meal, blood meal, or cracklings; vitamins, minerals, and amino acids; organic binders or adhesives; and chelating agents and preservatives.

In an embodiment feed pellets comprise components selected from a group consisting of proteins from plant meals such as meals derived from soy, corn and wheat, animal meals such as meat meal, blood meal and bone meal, and fishmeal; fish oil; vegetable oil (e.g. canola); binders; fillers; vitamins and minerals; and colourants. Antibiotic and other medical chemicals may be present but, if so, will be present in a reduced amount compared to a typical fish feed formulation, but are preferably absent.

In an embodiment the feed pellets comprise protein from fishmeal. In addition to its protein component, fishmeal also has a relatively high content of certain minerals, such as calcium and phosphorous, as well as certain vitamins, such as B-complex vitamins (e.g., choline, biotin and B12), and vitamins A and D. Industrial fishmeal usually also contains about 15% fish oil, which provides a source of important essential fatty acids.

In an embodiment the feed pellets comprise fish oil from the fishmeal and/or from other sources. Fish oil includes lipid-soluble vitamins (e.g., Vitamin A from fish liver oils) and certain preformed long chain polyunsaturated fatty acids (LC-PUFAs), such as arachidonic acid (ARA), eicosapentaenoic acid (EPA), and docosahexaenoic acid (DHA). Fish oil may be derived from wild caught fish or from other sources such as farmed fish or algal extracts.

The fillers and binders are used to bind the protein-rich ingredients together to improve stability in water. They are also useful in to improving the efficiency of the feed manufacturing process and to reduce feed wastage. Ingredients commonly used as binders in feed pellets include wheat gluten, sodium and calcium bentonites, lignosulfates, hemicellulose, carboxymethylcellulose, alginates, and guar gum. Binders such as bentonites, lignosulphonates, hemicellulose and carboxymethylcellulose reduce the frictional forces of the feed mixture as it passes through the pellet dies, thereby increasing the output and efficiency of the feed mill. Binders also increase the pellet hardness and reduce the formation of ‘fines’ during the pelleting process. Typical fillers include, for example, for example rice, soy, or wheat bran, rice, soy, or wheat flour, corn meal, rye, barley, sorghum, dextrose, sucrose, fructose, maltodextrin, starch or any combination thereof. Filler ingredients also often contain preservatives, such as, for example, ethoxyquin, which is often used as an anti-oxidant in fish feed.

Colorants are used in feed pellets for salmon to meet the consumer preference for red coloration in the flesh of the fish when it is consumed, and may be used in other fish species. Carotenoid pigments such as astaxanthin or canthaxanthin are often used as colorants. In order to meet the consumer preference for red coloration, salmonid flesh should contain at least 5-20 mg pigment per kg flesh. To achieve these levels at least 40-60 mg of canthaxanthin or 40-150 mg astaxanthin has to be added per kg of feed.

Vitamins and minerals may be added to the feed pellets. The person skilled in the art will appreciate that the identity of and the amount of vitamins and minerals required will vary among species. Typically, one or more vitamins selected from the group consisting of vitamin A, vitamin C, vitamin D3, vitamin E, pantothenic acid, niacin, inositol, vitamin B2, vitamin B6, thiamine, folic acid, biotin, vitamin B12 will be added. Typically, minerals selected from the group consisting of zinc, manganese, iodine, copper and potassium. Minerals may be added as salts, for example the abovementioned minerals may be added in the form of zinc sulfate, manganese sulfate, ethylene diamine dihydroiodide, copper sulfate and potassium sorbate, respectively, as would be well understood by the person skilled in the art. Amino acid supplements may also be included. Most commonly, the amino acids added are the essential amino acids for fish. In an embodiment one or more amino acids selected from the group consisting of threonine, valine, leucine, isoleucine, methionine, tryptophan, lysine, histidine, arginine and phenylalanine is added.

A typical feed formulation for fish in the grow out stage would generally include a protein source such as fish meal, defatted soybean meal, or poultry meal. It will also contain a carbohydrate source, with wheat meal, corn-starch, rice bran being popular options, and a lipid source including fish and vegetable oil. The feed will also contain a vitamin and mineral mix (vitamin A, C, D3, E, K3, B1, B3, B6, B5, B12, folic acid, inositol, biotin, copper sulfate, magnesium oxide, manganese sulfate, potassium iodide, iron sulphate, zinc oxide, dextrose and the antioxidant oxicap E2), mould inhibitor and amino acids supplements.

In embodiments Asparagopsis, on average, yielded about a 4.5-fold effect in immune stimulation when included orally as a raw ingredient at 3% w/w of the feed. The use seaweed of Asparagopsis species as an additive in fish feed provides an alternative or complement to other disease management strategies such as use of antibiotics.

The red seaweed, or an extract therefrom or the residue of the seaweed once extracted, may be formulated as a veterinary or pharmaceutical formulation. Such a formulation may be administered by any route suitable for the animal. By way of example, as appropriate, it may particularly be administered orally, intravenously, intramuscularly, cutaneously, subcutaneously, transmucosally (e.g. sublingually or buccally), rectally, transdermally, nasally, pulmonarily (e.g. tracheally or bronchially), or topically.

The red seaweed, or an extract therefrom or the residue of the seaweed once extracted, will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert and should have no detrimental side effects or toxicity under the conditions of use. Suitable pharmaceutical formulations may be found in, for example, Remington The Science and Practice of Pharmacy, 19th ed., Mack Printing Company, Easton, Pa. (1995). A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted veterinary or pharmaceutical practice.

In an embodiment, the red seaweed, an extract therefrom or the residue of the seaweed following an extraction process, is administered in conjunction with another seaweed that has effect in boosting the innate immune system. The seaweed may be selected from the group consisting of Sarconema sp., Gracilaria sp., Kappaphycus sp., Sargassum sp., Dictyota sp., Lobophora sp., Halimeda sp., Caulerpa sp., Ulva sp., Cyanobacteria such as Arthrospira/Spirulina sp. and microalgae such as Haematococcus pluvialis. Alternatively, an extract from another seaweed or the residue of the seaweed following an extraction process may be administered.

In an embodiment, innate immunity is increased by administering another seaweed, an extract therefrom or the residue of the seaweed following an extraction process. The seaweed may be selected from the group consisting of Sarconema sp., Gracilaria sp., Kappaphycus sp., Sargassum sp., Dictyota sp., Lobophora sp., Halimeda sp., Caulerpa sp., Ulva sp., Cyanobacteria such as Arthrospira/Spirulina sp. and microalgae such as Haematococcus pluvialis.

Seaweed of (Ulva sp. is effective in increasing innate immunity, particularly in increasing respiratory burst activity and lysozyme activity. Accordingly, innate immunity is increased by administering an effective amount of a green seaweed of Ulva species, an extract therefrom or the residue of the seaweed following an extraction process.

In an embodiment, respiratory burst activity is increased by administering another seaweed, an extract therefrom or the residue of the seaweed following an extraction process. The seaweed may be selected from the group consisting of Sarconema sp., Gracilaria sp., Kappaphycus sp., Sargassum sp., Dictyota sp., Lobophora sp., Halimeda sp., Caulerpa sp., Ulva sp., Cyanobacteria such as Arthrospiral/Spirulina sp. and Microalgae such as Haematococcus pluvialis.

Seaweed of Ulva sp. is effective in increasing respiratory burst activity. Accordingly, in an embodiment, respiratory burst activity is increased by administering to said animal an effective amount of the green seaweed of Ulva species, an extract therefrom or the residue of the seaweed following an extraction process.

In an embodiment, lysozyme activity is increased by administering another seaweed, an extract therefrom or the residue of the seaweed following an extraction process. The seaweed may be selected from the group consisting of Sarconema sp., Gracilaria sp., Kappaphycus sp., Sargassum sp., Dictyota sp., Lobophora sp., Halimeda sp., Caulerpa sp., Ulva sp., Cyanobacteria such as Arthrospira/Spirulina sp. and Microalgae such as Haematococcus pluvialis.

Seaweed of Ulva sp. is effective in increasing lysozyme activity. Accordingly, in an embodiment, lysozyme activity is increased by administering to said animal an effective amount of the green seaweed of Ulva species, an extract therefrom or the residue of the seaweed following an extraction process

In an embodiment, the red seaweed, an extract therefrom or the residue of the seaweed following an extraction process, is administered in conjunction with another immunostimulant. In an embodiment the immunostimulant is hydrolysed yeast (Saccharomyces cerevisiae) sold as Hilyses® (York Ag Products Inc). In an embodiment, the immunostimulant is sodium alginate.

The person skilled in the art will appreciate that different algae, extracts residues and/or commercial immunostimulants may be administered sequentially, simultaneously or concomitantly This may involve administration of separate formulations, be they animal feeds or pharmaceutical or veterinary formulations, with one containing the red seaweed, and another containing the other active ingredient. These may be provided together as a kit of parts with instructions for use. Alternatively, it might involve administration of a single animal feed, pharmaceutical or veterinary formulation containing the red seaweed and the other component or components.

### EXAMPLES

Hereinafter, embodiments of the invention are illustrated in more detail with reference to the following examples. However, the present disclosure is not limited thereto. Furthermore, what is not described in this disclosure may be sufficiently understood by those who have knowledge in this field and will not be illustrated herein.

Eleven species of seaweed (marine macroalgae) from three taxonomic groups (green, brown and red) were evaluated against two immunostimulant target species of microalgae (the astaxanthin-containing alga Haematococcus and Spirulina (now Arthrospira)), two positive controls (brown seaweed-derived sodium alginate and β-glucan-rich Hilysese) and a procedural control (no additive). All treatments were delivered as a 3% w/w dietary inclusion in the fish feed. The extent of change in four immune parameters, ACH50, phagocytosis, lysozyme activity and respiratory burst, that assess the innate immunity of the fish were measured.

### Example 1 Collection of Seaweed and Preparation of Fish Feed

The origin, treatment and processing of the seaweeds used in this study is summarised in Table 1. Two positive controls were used in this study namely Hilyses®, a hydrolysed yeast culture derived from the sugarcane fermentation process and sodium alginate, the anionic polysaccharide extracted from brown seaweeds. In addition, the cyanobacteria Spirulina (Swisse high strength organic Spirulina) and the microalgae Haematococcus pluvialis were also included as positive control as often used in fish studies. All seaweeds were rinsed with salt water to remove sand and biological contaminants. They were then spun to get rid of excess water and frozen at −80° C. before being processed in a freeze dryer (Thermo Savant model MODULYOD-230) for at least 3 days at approximately −44° C. and 206 mbar. Once dried, all seaweed species were vacuum sealed in individual bags with desiccant and stored at −20° C. until needed. The control diet was produced based on the commercial pellet Native (Ridley Aquafeeds Ltd). The Native pellets were first powdered and then warm water was slowly added in a blender (Hobart A120) for approximately 10 min to produce a stiff dough. The dough was extruded through a 4 mm die on trays which were then oven dried overnight at 50° C. Once dried the feed was crumbled and packaged in airtight bags, subsequently stored at 4° C. for the duration of the trial. The experimental diets were made in the same manner but received the powdered and 300 μm sieved ingredients (Table 1) prior to adding the water during the blending step.

TABLE 1
List of ingredients trialled

| Species | Morphology | Processing |
|---|---|---|
| Sarconema sp. | Soft fleshy, branching | Freeze dried |
| Gracilaria sp. | Soft fleshy, branching | Freeze dried |
| Kappaphycus sp. | Rigid, branching | Sun dried |
| Asparagopsis sp. | Soft, fleshy | Freeze dried |
| Sargassum sp. | Fleshy, turgid | Freeze dried |
| Dictyota sp. | Fine, dichotomously branching | Freeze dried |
| Lobophora sp. | Tough, leathery, thallus | Freeze dried |
| Halimeda sp. | Hard, calcifying | Freeze dried |
| Caulerpa sp. | Soft, fleshy | Freeze dried |
| Ulva sp. | Single cell thick blade | Freeze dried |
| Cyanobacteria | | |
| Spirulina sp. | Marine filamentous blue- | |
| | green algae | |
| Microalgae | | |
| Haematococcus | Unicellular, spherical cysts | Refractance |
| pluvialis | (cracked biomass) | window dried |
| Commercial | | |
| Immunostimulants | | |
| Hilyses ® | Hydrolysed yeast | Dried |
| | (Saccharomyces | |
| | cerevisiae) | |
| Sodium alginate | The sodium salt of alginic | Dried |
| | acid which is extracted from | |
| | the cell wall of brown algae | |

### Example 2 Fish Feeding

The juvenile rabbitfish, Siganus fuscescens, were captured using a drag net (15 m long by 2.1 m deep with a 2.5 cm mesh size). All fish were collected at Moffat Beach, Queensland Australia (26°47′21.7″S 153°08′36.0″E) on rocky reefs off the beach and transferred to the Bribie Island Research Centre (BIRC) in an oxygenated 500 L fish carrier. Once at BIRC, they received a hydrogen peroxide bath (200 mg/l for 30 min) to rid the fish of potential external pathogens and parasites. After treatment, the fish were transferred in three 1000 L fibreglass tanks where they were acclimatised and fed the control diet for two weeks. For the screening trial, 144 fish in total made up of three groups of 48 fish (small, medium and large). The initial fish weight for each group was 85.83±7.85 g, 112.60±8.17 g and 150.59±14.59 g for the small, medium and large fish groups respectively. The fish were randomly allocated into 48 plastic tanks (55 l) at a rate of 3 fish per tank, with one fish from each group. One replicate tank per treatment was stocked each day over three days. The diets were hand fed at 3% w/w body weight twice a day (10:00 a.m. and 3:00 p.m.). During the trials, the water temperature was maintained at 27° C. and pH in a range of 7.9 to 8.1. The system was operated as flow through using seawater pumped approximately 300 m off the beach adjacent to the station. The seawater is then pumped through a series of 16 spin disk filters (40 μm) and 10 multimedia filters (˜10-15 μm), after which it receives ozone treatment from two 100 gO₃/h generator units (WEDECO OCS-GSO30). The ozone treated seawater is then pumped via ultra violet filters, providing 80 mJ/cm², to two 4×2.2 m granular activated carbon vessels for a contact time of >9 mins to remove unwanted by-products from the ozone treatment. Finally, the seawater is pumped to a header tank, which fed directly into a pipe system delivering treated seawater to this experiment. The system was in a temperature and light controlled room kept at 24-26° C. and on a 24:0 L:D light regime.

### Example 3 Sample Collection and Preparation

After a 24 h fasting period the fish from one replicate tank per treatment was harvested each day over a three days period. Blood samples were collected at the end of each feeding trial by sampling the caudal vein from 3 fish tank-1 using a 1 ml syringe. The samples were immediately withdrawn into Eppendorf tubes one with and one without heparin. The tube with heparin was stored at 4° C. until analysed (<24 h). The tube without heparin was allowed to clot for 1 h at room temperature and 8 h at 4° C. and subsequently centrifuged at 1500×g for 5 min at 4° C. The separated serum was then collected, aliquoted into 1.5 ml Eppendorf tubes and stored at −80° C. until analysed. The heparinised blood samples were analysed the same day for cell counts.

The fish were then weighed and their liver dissected out to calculate their hepatosomatic index (HIS; see equation below). The whole gut from each fish was aseptically removed and placed in a 50 ml centrifuge tube before being frozen and stored at −80° C. until further processing could occur.

### Example 4 Cell Counts

The tubes containing the heparinised blood were inverted gently and diluted 1:500 in phosphate buffered saline (137 mM NaCl, 10 mM Phosphate, 2.7 mM KCl, pH 7.4) containing 1% of formalin (37%). The diluted samples were then gently inverted and 10 μl were transferred to Neubauer improved cell counting chamber to enumerate the erythrocytes and leukocytes on a light microscope (×100; Nikon Eclipse E200).

The number of cells per μl of blood was the results of triplicate counts and calculated as follow:

Cell/μl=(number of cells×dilution factor (500))/volume of haemocytometer (0.1 μl)

### Example 5 Immunological Assays

i. Phagocytic Activity

The phagocytic activity assay was based on the method from Anderson and Siwiski (1995). The phagocytic cells and the phagocytic index was determined using 1 μm fluorescent beads (Sigma). Fifty microliters of a heparinised blood sample was placed in the wells of microtiter plate followed by 50 μl of 1×10⁷ 1 μm fluorescent beads (Sigma) suspended in phosphate buffered saline (pH 7.2). The mixture was mixed thoroughly and incubated for 1 h at room temperature. Five microliters were then taken out onto a glass slide to prepare a smear. The smear was air dried (10 min) and then fixed with 95% methanol. Once the methanol evaporated, the smear was stained with Giemsa stain for 30 minutes. The number of phagocytes, phagocytising cells and the number of engulfed fluorescent latex beads was counted using an epifluorescence microscope (Nikon Eclipse Ti-U with X-Cite series 120 Q from Lumen Dynamics). The phagocytic activity (PA) and phagocytic index (PI) were calculated as follows:

PA=number of phagocyte containing beads/total number of phagocytes

PI=total number of beads engulfed by phagocytes/total number of phagocyte-containing beads
ii. Respiratory Burst Activity

The production of reactive oxygen species by leukocytes was measured using nitrotetrazolium blue chloride (NBT, Sigma) and triggered using phorbol 12-myristate 13-acetate (PMA, Sigma) following the method from Secombes (1990) and subsequently modified by Stasiack and Bauman (1996) (Secombes, 1990; Stasiak & Baumann, 1996). Briefly, 50 μl of blood samples were loaded in ‘flat bottom’ microtiter plates and incubated at 27° C. for 1 h to allow adhesion of cells. The supernatant was decanted and the wells were washed thrice with PBS. Fifty microliters of 0.2% NBT containing 200 ng/ml of PMA was loaded in the wells and incubated for 1 h at 27° C. The cells were then fixed using 100% methanol for 2 min and washed thrice using 70% methanol. The plates were air-dried after which 60 μl of 2N potassium hydroxide and 70 μl of dimethyl sulfoxide were added to all well to dissolve the formazan blue precipitate formed by the reactive oxygen species. Finally, the optical density of each well was measured at room temperature and recorded in an EnSpire multimode plate reader (PerkinElmer) at 540 nm.
iii. Serum Lysozyme Activity

Serum lysozyme activity was determined using the turbidometric assay, which gives a direct measure of lysozyme activity, calculated from a Hen Egg White Lysozyme (HEWL) internal standard (Ellis, 1990) (Ellis, 1990). Briefly, lyophilized Micrococcus lysodeikticus (75 mg) was rehydrated and suspended in 100 ml of buffer (0.05M Na₂HPO₄, pH 6.2) to achieve a 0.075% w/v concentration. Flat bottom microtiter plates were dosed with 140 μl of buffer and 10 μl of thawed serum sample. The plate was then shaken and absorbance was measured at 450 nm at 0.5 and 4.5 min. One unit of lysozyme was defined as a decrease of 0.001 in absorbance over that period.
iv. Haemolytic Activity of the Alternative Complement Pathway

The haemolytic activity of the alternative complement pathway (ACH50) was determined as the method described by Sunyer and Tort (1995). Briefly, rabbit red blood cells (R-RBC) were washed thrice in Hanks buffered saline solution (HBSS) supplemented with 7 mM MgCl₂ and 10 mM EGTA (HBSS-Mg-EGTA) with successive centrifugation (2000 rpm for 5 min at 4° C.). The R-RBC were rinsed three times with HBSS-Mg-EGTA for 1 min at 1000 rpm and made up to 3% volume in the same buffer. In 96-well plates, 20 μl of test serum was diluted with 30 μl of HBSS-Mg-EGTA and four-fold serial dilutions were made to achieve dilutions ranging from 0.15-10.00%.

However, some samples required further dilution in this instance the four-fold serial dilution went from Subsequently, 20 μl of R-RBC suspension was added to each tube and incubated for 100 min at 27° C. with occasional shaking. Three replicate negative control were also made for the 0% and 100% lysis of R-RBC by adding 20 μl of the R-RBC suspension to 120 μl of HBSS-Mg-EGTA and distilled water respectively. After incubation, the plates were centrifuged at 2000 rpm for 2 min and 50 μl of supernatant of each dilution was then transferred to a new microtiter plate and read at 540 nm. The degree of haemolysis was calculated by dividing the corrected absorbance 540 value by the 100% haemolysis control. The reciprocal of the serum dilution giving 50% haemolysis was used as the ACH50 titre (units/ml).

### Example 5 Salmon Methods and Measurement of Immune Parameters

Asparagopsis taxiformis was collected from Moffat Beach, Queensland Australia (26°47′21.7″S 153°08′36.0″E). The seaweed was cleaned using seawater to remove sand and epiphytes before being spun to remove excess salt water. Following this the seaweed was frozen at −80° C. before being processed in a freeze dryer (Thermo Savant model MODULYOD-230) for at least 3 days at approximately −44° C. and 206 mbar. Once dried, the seaweed was powdered and kept in a vacuumed sealed bag in the −80° C. until future use. The control diet was produced based on the commercial Nutra Supreme-RC (Skretting Ltd). The Skretting pellets were first powdered and then warm water was slowly added in a blender (Hobart A120) for approximately 10 min to produce a stiff dough. The dough was extruded through a 2 mm die on trays which were then dried in a fan driver food dehydrator (Sunbeam) at room temperature for 12 h. Once dried the feed was packaged in airtight bags and subsequently stored at 4° C. for the duration of the trial. The experimental diets were made in the same manner but received the powdered and 300 μm sieved seaweed or the seaweed extract prior to adding the water during the blending step.

The seaweed extract was made using 150 g of freeze dried A. taxiformis, which was extracted 4 times over 12 h in 500 ml of methanol. The 1 l extract rich methanol was then filtered prior to being slowly evaporated using a rotary evaporator with the extract sitting in a 30° C. bain-marie. Once the methanol fully evaporated the extract was resuspended in 400 ml of deionised water and 100 ml of hexane. The extract for the two extract treatments (3% and 6%) was added at the equivalent proportion of whole seaweed. A positive control lipopolysaccharide (LPS from Escherichia coli, purchased from Sigma) was added at 0.01% w/w into the feed.

The salmon fry, Salmo salar (5 g), were shipped from a hatchery in Tasmania to the Bribie Island Research Centre (B1RC). Once at B1RC, they were spread in between two 1000 L fiberglass conical tanks where they remain for and acclimation period of 6 days. The fish were then randomly allocated into 50 plastic tanks (55 l) at a rate of 18 fish per tank. The diets were hand fed to satiation twice a day (10:00 a.m. and 3:00 p.m.). During the trials, the water temperature was maintained at 15° C. by a heat pump (Oasis C16) and pH in a range of 7.0 to 7.8. The system was operated as a recirculating aquaculture system using dechlorinated town water and comprised of two Waterco C50 bag filters in parallel (50 μm bags) followed by a Micron S602e sand-filter. The system was in a temperature and light controlled room kept at 18° C. and on a 12:12 L:D (08:00-20:00) light regime with a 30 min ramp up/down period.

After two weeks on the treatment and control diets the fish were fasted for 24 h prior to sampling. Once fasted, 3 fish per tank were randomly sampled to draw blood (heparinised using lithium heparin or without heparin for serum) for the four innate immune parameters (Example 5). These fish were weighed and had their liver and head kidney removed and placed in RNA later for gene expression (Example 7). All remaining fish in the replicate tanks were weighed and returned to their tanks for another two weeks on the treatment diets. This was again followed by a 24 h fasting period after which the same samples (Example 5) were taken.

Immune parameters were measured as per Example 5. Gene expression was measured as per Example 7.

### Example 6 Chemical Analysis of the Methanolic Extract of Asparagopsis

The methanolic extract was created as per Example 5. The key natural products were analysed using Gas Chromatography-Mass Spectrometry. The method for analysis was as follows:

In order to make the seaweed extract treatments used in feed, a sample of freeze-dried Asparagopsis taxiformis was extracted in methanol in a round-bottom flask and the methanol driven-off under nitrogen stream. The residue was subsequently extracted in methanol and the process repeated for a total of 4 extractions. The extracts were combined, filtered and then subjected to rotary evaporation under vacuum until all methanol was driven-off. The approximate recovery of extract was 20% by mass of the original freeze-dried sample of Asparagopsis taxiformis. The extract was reconstituted in methanol with ethyl benzoate as internal standard, filtered and vialled for Gas Chromatography-Mass Spectrometry analysis. A portion of the original freeze-dried sample of Asparagopsis taxiformis (whole seaweed treatment) was taken for direct analysis by Gas Chromatography-Mass Spectrometry. For the whole seaweed treatment, the sample was directly extracted in methanol with ethyl benzoate as an internal standard, filtered and vialled for Gas Chromatography-Mass Spectrometry analysis.

Gas Chromatography-Mass Spectrometry was performed on a Perkin Elmer Glarus SQ8S fitted with a DB-5 column (Perkin Elmer Elite-5MS, 30.0 m×0.25 mm, 025 μm). Injections (1.0 μL) were introduced with a 50:1 split ratio with a sample rate of 1.56250 pts/sec. The GC was held at 40.0° C. for 1 min, ramped at 20.0° C. min-1 to 250.0° C. and held for 0 min followed by a 0.5 min equilibration time prior to the next injection. Helium was used as the carrier gas with a flow rate of 1 mL/1 min. Mass spectrometry was performed on a Perkin Elmer Glarus 580 across a weight range of 50-340 m/z. Analysis occurred from 3.0-12.0 min with a scan rate of 0.3 s. Compounds were identified by referencing mass spectral chromatographs to the NIST library. G.C. confidence intervals were then averaged across samples as well as within samples using different areas of the peak and subtraction of background ion profiles. Relative quantitation was achieved by comparison of peak area ratios (as determined using supplied TurboMass software) of compound to internal standard (equivalent to parts per million or compound (mg)/solvent (L)) which were then evaluated to give compound (g)/algae material (g).

In the table following, the percentages represent the relative abundance of the top 10 compounds detected in the whole Asparagopsis seaweed and in the extract, summing to 100%. Note that compound names denoted with (*) represent unique compounds not found in top 10 of both samples (unidentified compounds are indicated as compounds 1-6 with most likely halogenation). Note that there are up to 4 unique compounds in the list of the top 10 most abundant halogenated compounds in whole seaweed, compared to up to 3 unique compounds in the list of the top 10 most abundant halogenated compounds in the solvent extract.

TABLE 2
Kev compounds detected in Asparagopsis
and its methanolic extract

| | Whole Asparagopsis | | Solvent extract | |
|---|---|---|---|---|
| # | Compound | % | Compound | % |
| 1 | Bromoform | 68% | Tribrominated | 32% |
| | | | compound 1 | |
| 2 | Tribrominated | 18% | Dibromoacetic acid | 27% |
| | compound 1 | | methyl ester * | |
| 3 | Dibromoiodomethane | 4% | Bromoform | 9% |
| 4 | Dibromoacetic | 2% | Dibrominated | 6% |
| | acid * | | compound 6 | |
| 5 | Halogenated | 2% | Tribromoacetic acid | 6% |
| | compound 2 | | methyl ester * | |
| 6 | Dibromochloro- | 2% | Dibromoiodomethane | 5% |
| | methane* | | | |
| 7 | Dibrominated & | 1% | Dibromobutenedioic | 4% |
| | chlorinated | | acid * | |
| | compound 3 * | | | |
| 8 | Dibrominated | 1% | Halogenated | 4% |
| | compound 4 | | compound 2 | |
| 9 | Tetra- | 1% | Dibrominated | 4% |
| | bromomethane * | | compound 5 | |
| 10 | Dibrominated | 1% | Bromoacetic | 3% |
| | compound 5 | | acid | |
| | % total | 100% | % total | 100% |

### Example 7 Gene Expression of Immune and Stress Related Genes in Salmon

All RNA extraction was performed using RNeasy mini kit (Qiagen, Australia), as per manufacturer's instructions. For the elimination of DNA, DNase I (Sigma-Aldrich, Australia) was used, as per manufacturer's instructions. Prior to reverse transcription, extracted RNA was quantified using Qubit, and for each reverse transcription reaction we used a total 1000 ng RNA in reaction. Reverse transcription was performed using iScript reverse transcription supermix (Bio-Rad, Australia), as per manufacturer's instructions. cDNA was then stored at −20° C. until utilised in qRT-PCR for gene expression.

cDNA, extracted from livers and head kidneys, was used to measure the level of salmon gene expression using quantitative real-time PCR (qPCR). Briefly, the qPCRs were performed in a final volume of 20 ul, including 10 ul iTaq master mix (Biorad), 0.75 ul of 10 uM each of forward and reverse primer (0.375 uM primer concentration in reaction) (Easy oligos, Sigma-Aldrich), 6.5 ul PCR grade water and 2 ul template cDNA. Cycling conditions consisted of 3 min at 95° C., followed by 35 cycles of 15 sec at 95° C., 25 sec at 62° C. and 30 sec extension at 72° C., followed by melt curve analysis (65° C. to 95° C. in 0.5° C. increments). Samples were tested in duplicates, and negative controls (MilliQ water and no transcribed sample) were included in each assay. In each assay, a CT value was recorded for each sample. Each gene amplicons were characterised with a high-resolution melt (HRM). Samples with >Ct 35 and below the signature melt threshold were conservatively considered negative.

Gene expression of IFN-γ, IL-1β, Lys, C3, IFNγ and HSP was measured in both the liver and head kidney and compared to the house-keeping gene EF1. Salmon gene expressions were normalised to EF1 using the 2-ΔΔCT equation (where CT is threshold cycle): ΔCT=(CT of cytokine gene−CT of EF1gene) and ΔΔCT=(ΔCT of stimulated sample−ΔCT of unstimulated sample)

TABLE 3
Genes targeted in this study

| Gene | Forward primer | Reverse primer |
|---|---|---|
| name | sequence (5′ to 3) | sequence (5′ to 3) |
| EF1 | CCCCTCCAGGACGTTTACAAA | CACACGGCCCACAGGTACA |
| IL-1β | ATGCGTCACATTGCCAAC | GGTCCTTGTCCTTGAACTCG |
| Lys | CTACAATACCCAGGCCACCAA | GTCACACCAGTAGCGGCTGTT |
| IFNy | CCGTACACCGATTGAGGACT | GGGCTTGCCGTCTCTTCC |
| C3 | GAAGAAAGGTGAGCCAGATG | TGTGTGTGTCGTCAGCTTCG |
| HSP | CCCCTGTCCCTGGGTATTG | CACCAGGCTGGTTGTCTGAGT |

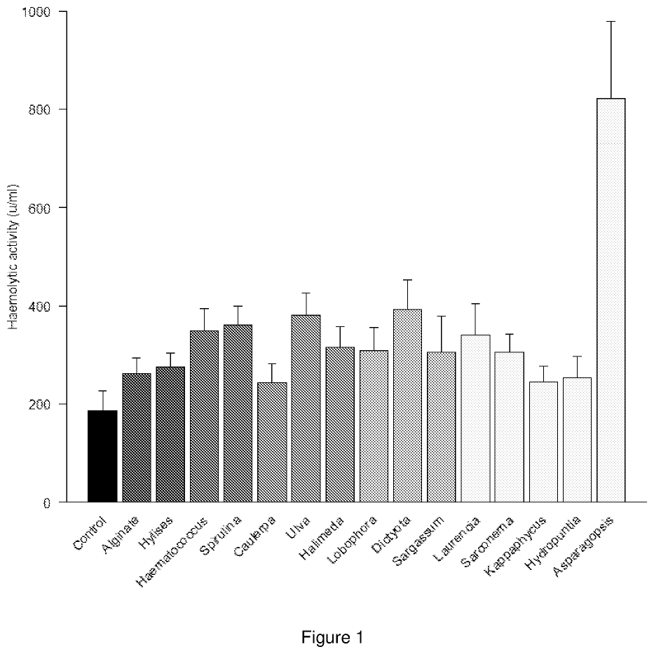

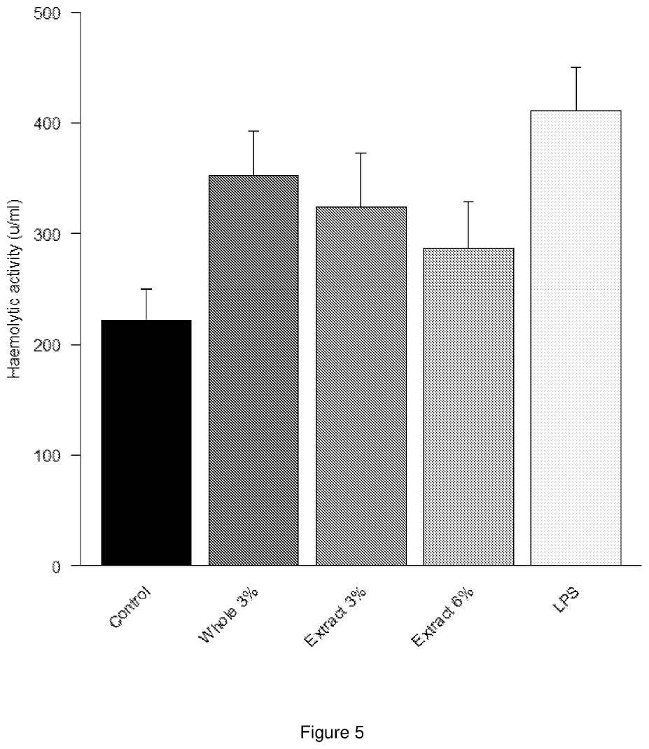

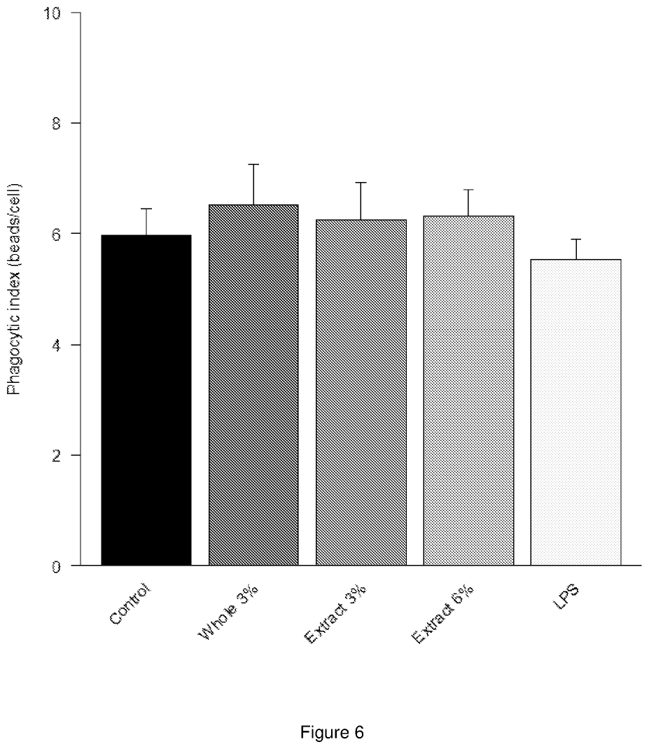

We found specific activity of Asparagopsis with one immune parameter—serum haemolytic activity, also known as ACH_{50,} was approximately 400% of the level of the control. Out of the 15 other treatments tested, the next closest to Asparagopsis was +150% of control (FIG. 1). Essentially, the Asparagopsis effect was double the next most effective treatment. Asparagopsis whole and extracts were demonstrated to elevate ACH₅₀ in salmon (FIG. 5).

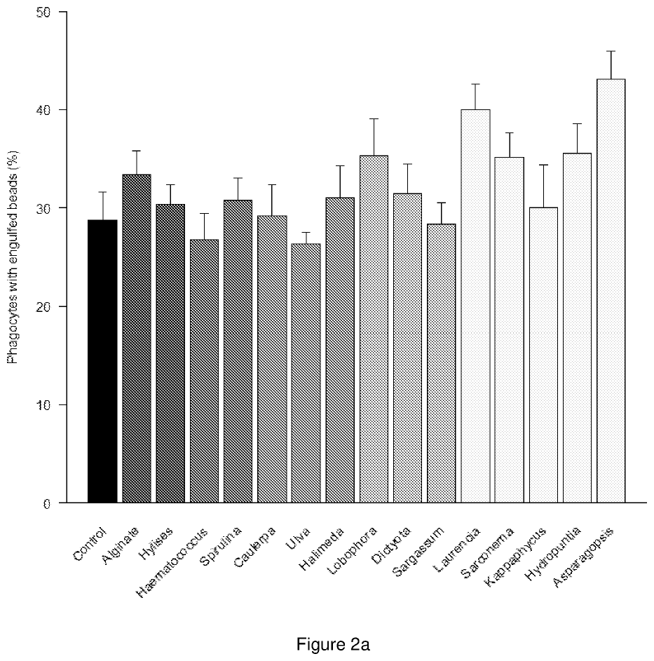

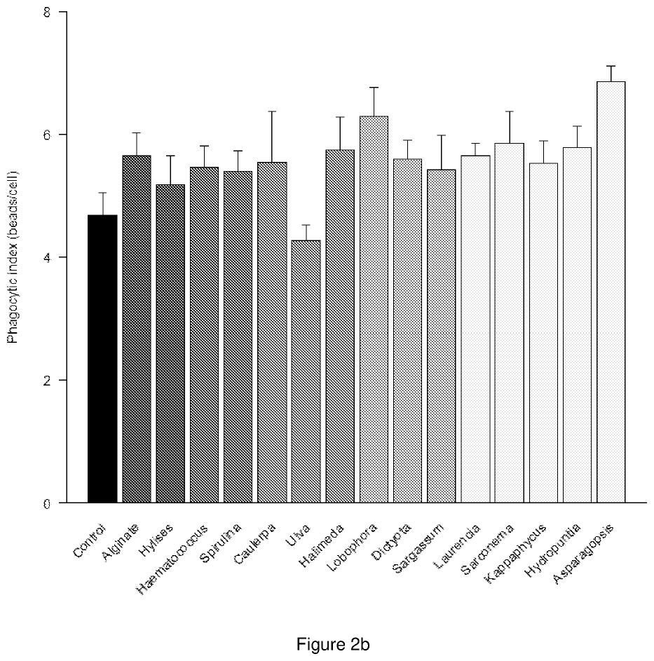

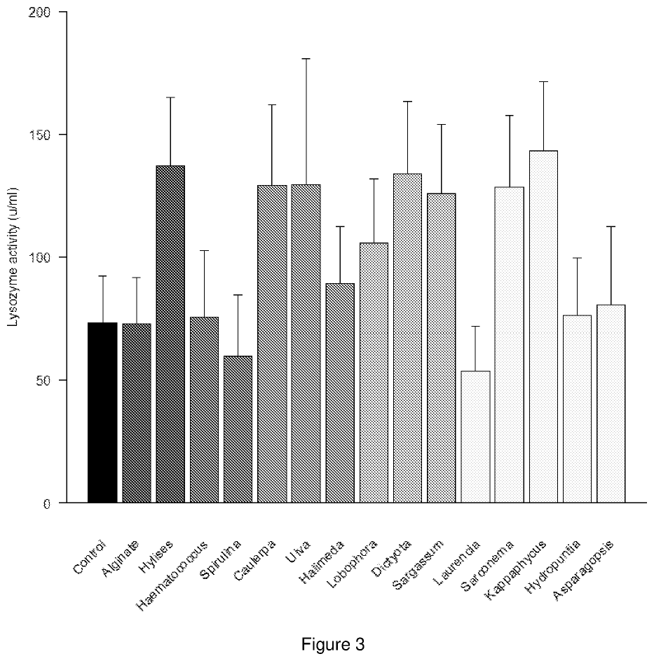

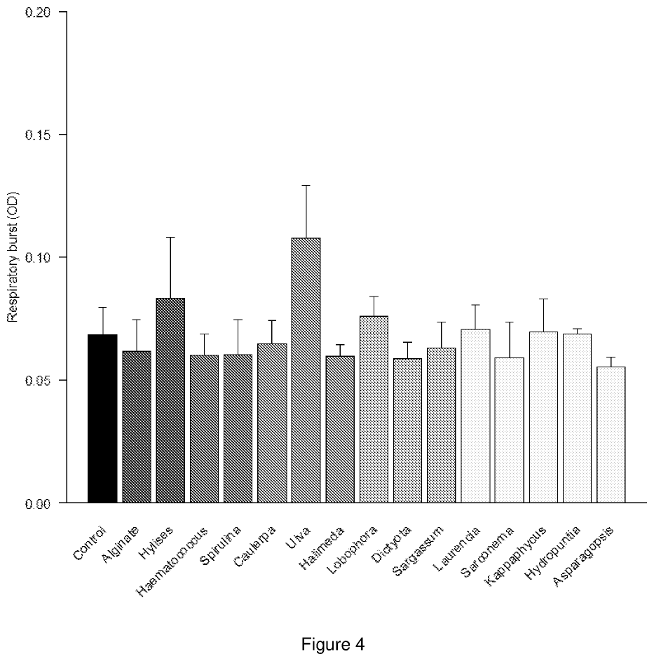

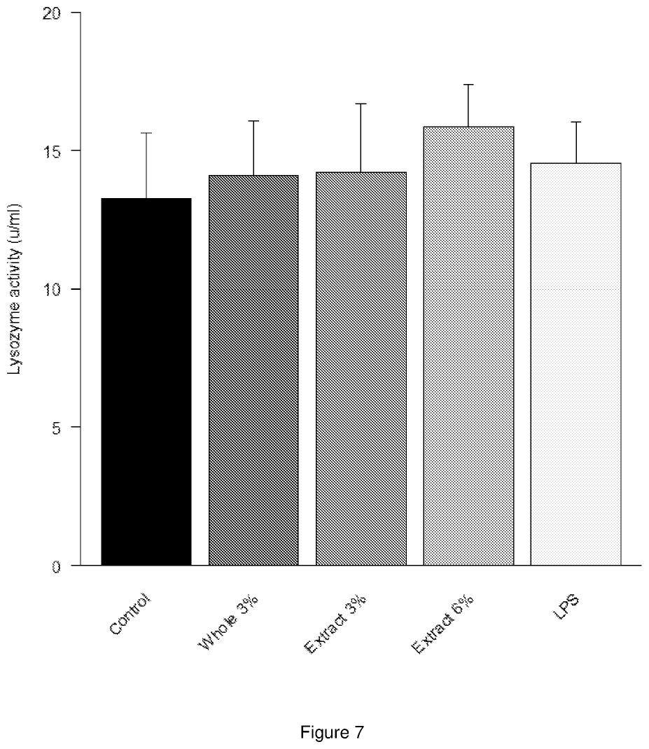

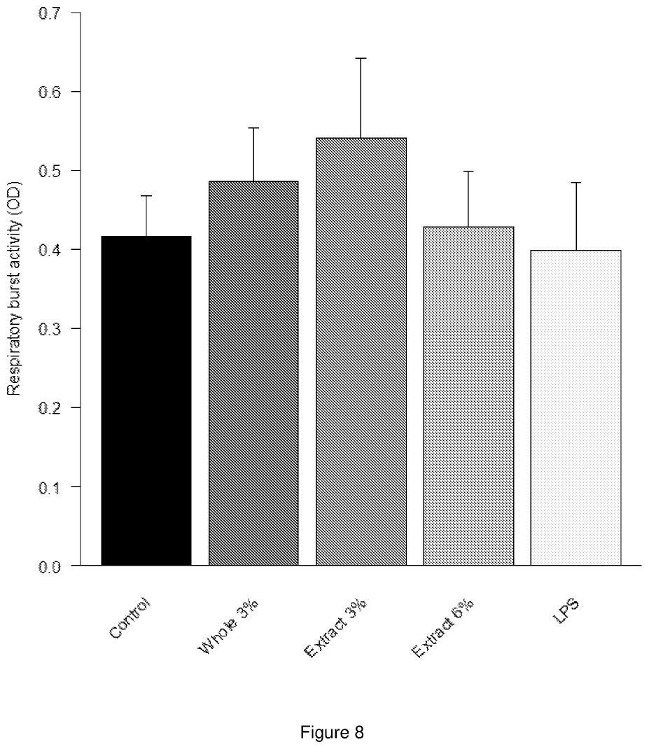

Asparagopsis was shown in rabbiffish the highest effect for the second immune parameter—both measures of phagocytosis (FIG. 2a and b). No unique effects of Asparagopsis were observed on the two other immune parameters: lysozyme activity—(FIG. 3) and respiratory burst activity—(FIG. 4). Asparagopsis extracts were demonstrated to elevate lysozyme activity (FIG. 7) and respiratory burst activity (FIG. 8) in salmon relative to controls.

The green seaweed Ulva was shown in rabbitfish to have the highest effect for the two other immune parameters: lysozyme activity (FIG. 3) and respiratory burst activity (FIG. 4).

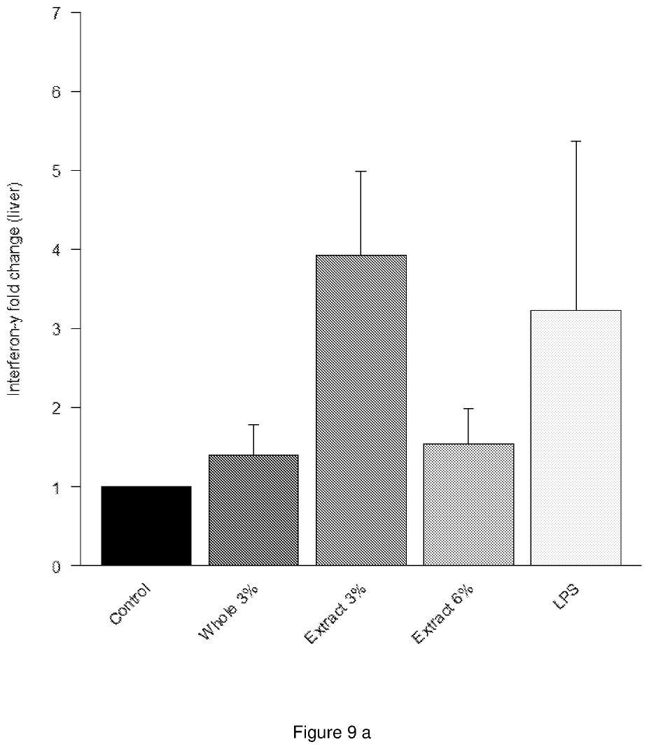

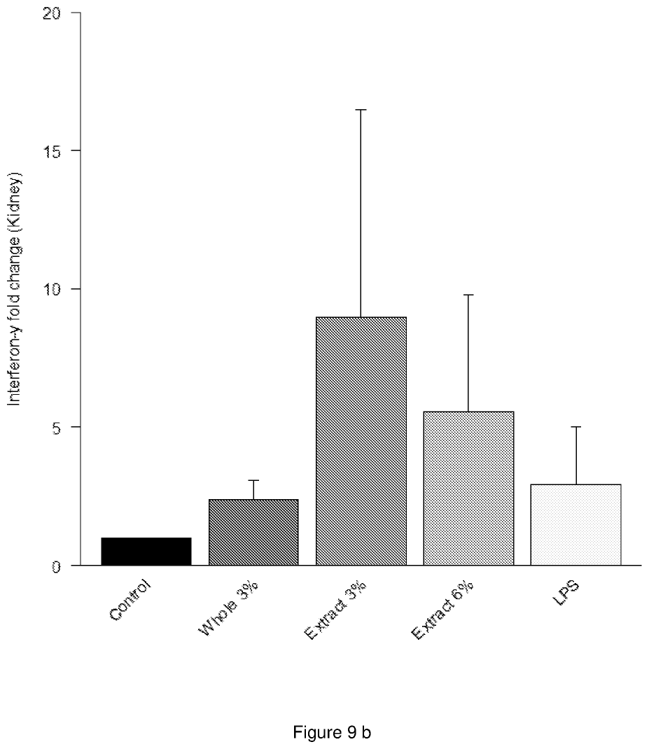

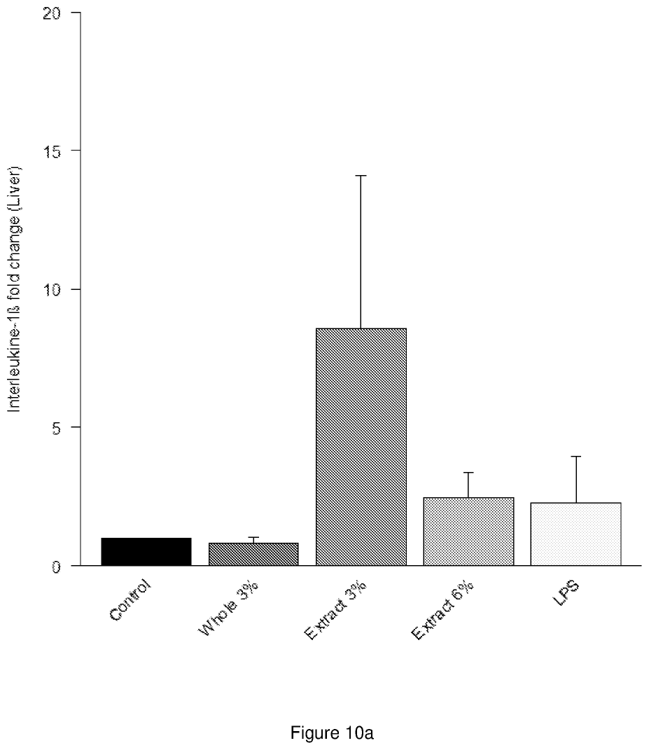

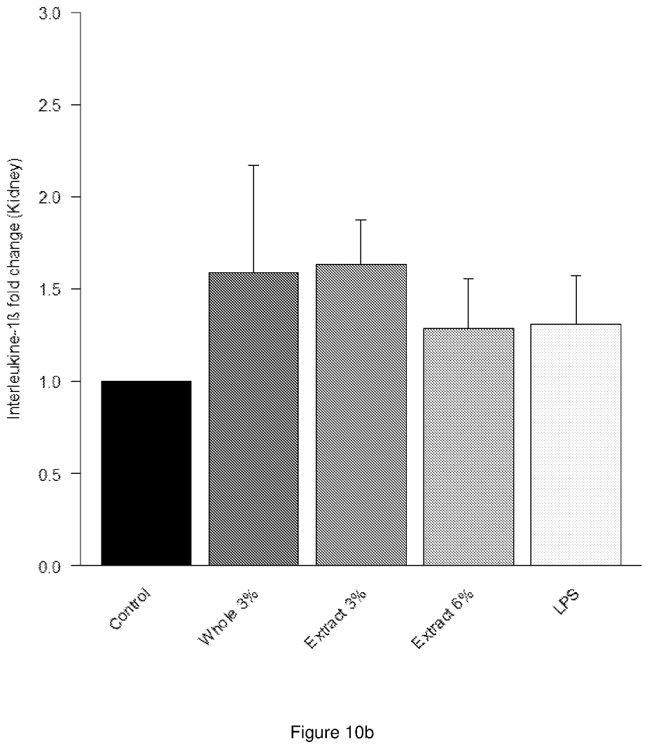

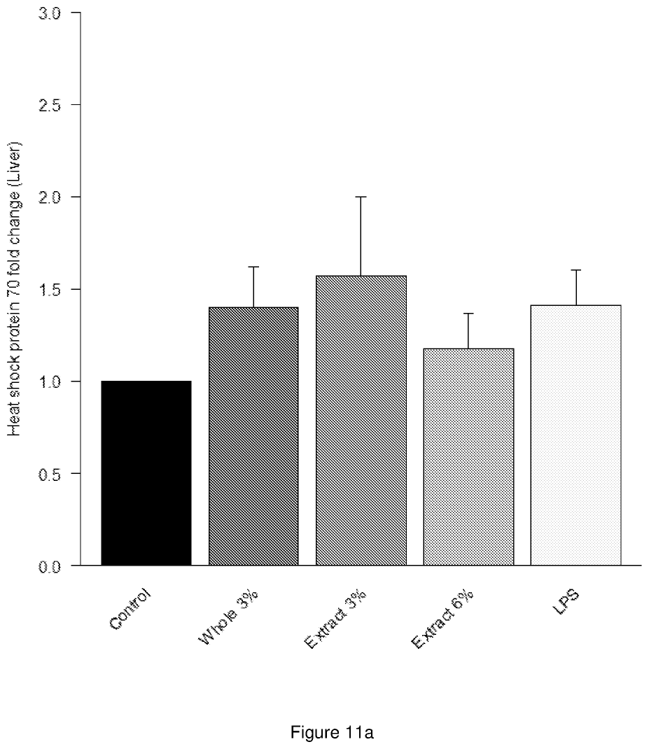

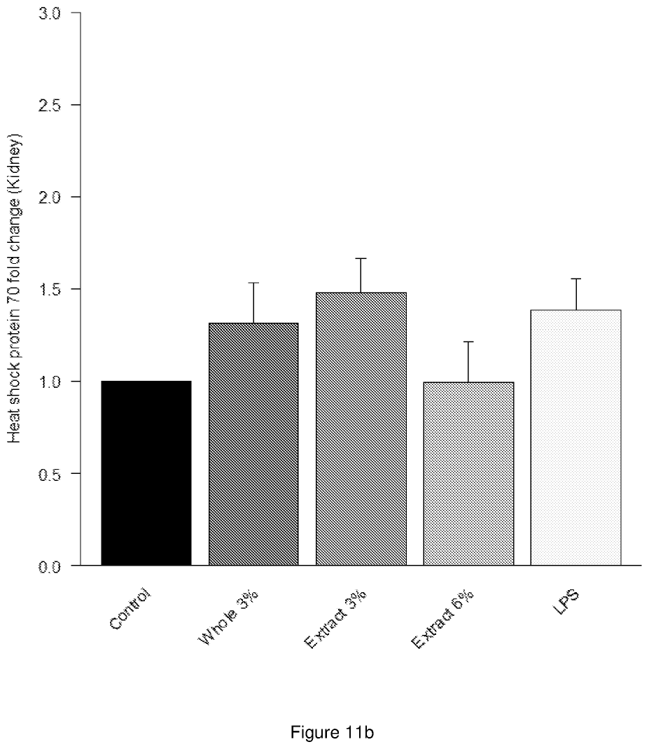

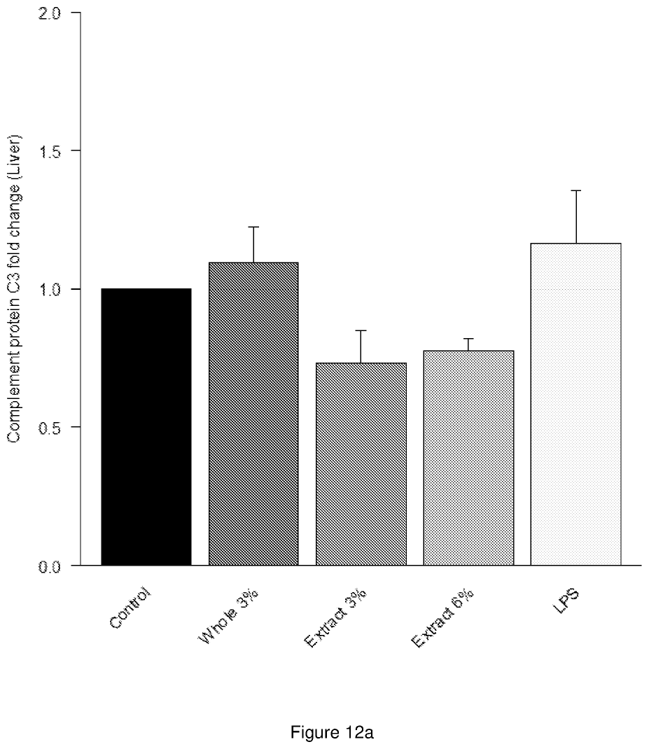

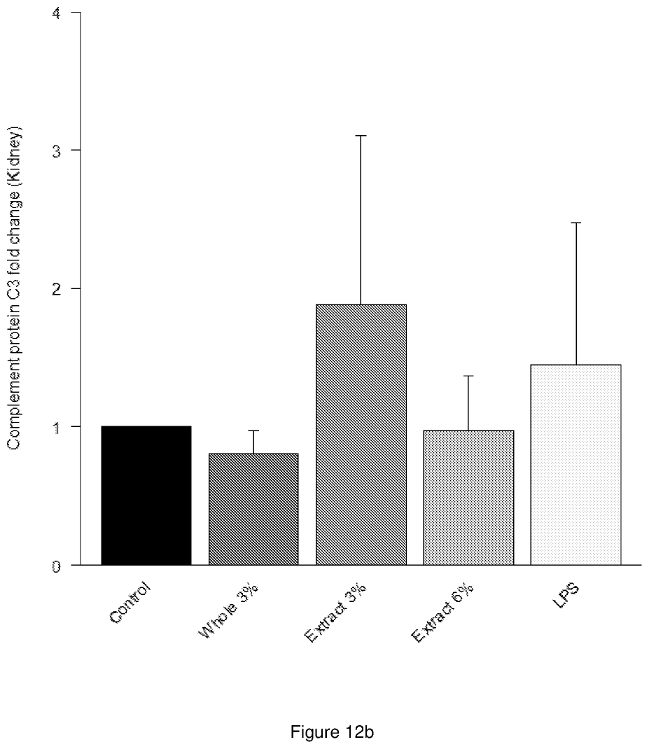

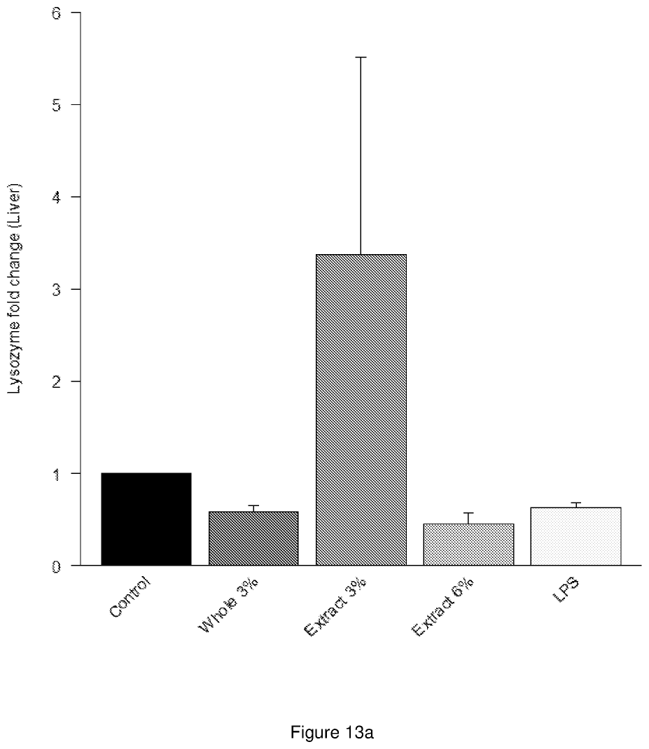

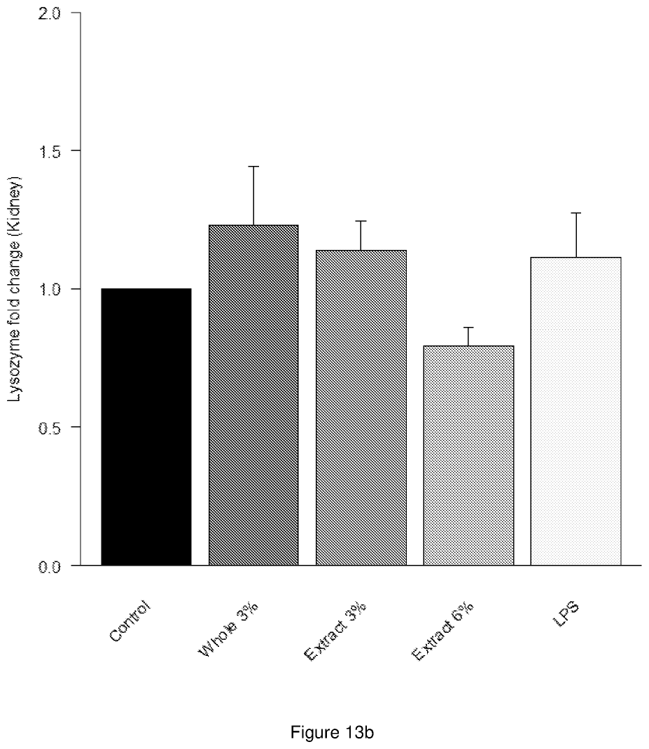

Asparagopsis extract at equivalent of whole seaweed at 3% inclusion was demonstrated to elevate gene expression of Interferon-γ (in liver and kidney, IFNγ, FIG. 9), Interleukine-1β (in liver and kidney, IL-1β, FIG. 10), Heat shock protein-70 (in liver and kidney, HSP F2, FIG. 11), Complement protein-3 (in kidney, C3, FIG. 12) and Lysozyme-C (in liver, Lys, FIG. 13) in salmon relative to control (Y-axis=1). Essentially, the Asparagopsis effect was more than double the next most effective treatment for interleukine-1β in liver and Lysozyme-C in liver.

Asparagopsis whole at 3% inclusion was demonstrated to elevate gene expression of Lysozyme-C in kidney (FIG. 13) in salmon relative to control (Y-axis=1).

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

### REFERENCES

References cited herein are incorporated herein by this reference and are as follows: Anderson D. Siwicki A (1995) Basic haematology and serology for fish health programs. In: Diseases in Asian Aquaculture II (ed. by Shariff M, Arthur J R, Subasinghe R P), Fish Health Section, Asian Fisheries Society, Philippines, pp. 185-202.

Bouwhuis, M., McDonnell, M., Sweeney, T., Mukhopadhya, A., O'Shea, C., O'Doherty, J., 2017. Seaweed extracts and galacto-oligosaccharides improve intestinal health in pigs following Salmonella typhimurium challenge. animal. 11, 1488-1496.

Boshra H, Li J, Sunyer J (2006) Recent advances on the complement system of teleost fish. Fish & shellfish immunology, 20, 239-262.

Bouwhuis M A, Sweeney T, Mukhopadhya A, McDonnell M J, O'Doherty J V (2017) Maternal laminarin supplementation decreases Salmonella typhimurium shedding and improves intestinal health in piglets following an experimental challenge with S. typhimurium post-weaning. Animal Feed Science and Technology, 223, 156-168.

Bricknell I, Dalmo RA (2005) The use of immunostimulants in fish larval aquaculture. Fish & Shellfish Immunology, 19, 457-472.

Burridge, L., Weis, J. S., Cabello, F., Pizarro, J., Bostick, K., 2010. Chemical use in salmon aquaculture: A review of current practices and possible environmental effects. Aquaculture. 306, 7-23.

Cabrita A R J, Correia A, Rodrigues A R, Cortez P P, Vilanova M, Fonseca A J M (2017) Assessing in vivo digestibility and effects on immune system of sheep fed alfalfa hay supplemented with a fixed amount of Ulva rigida and Gracilaria vermiculophylla. Journal of Applied Phycology, 29, 1057-1067.

Chang, Q., Wang, W., Regev-Yochay, G., Lipsitch, M., Hanage, W. P., 2015. Antibiotics in agriculture and the risk to human health: how worried should we be? Evolutionary Applications. 8, 240-247.

Cordero H, Guardiola F A, Tapia-Paniagua S T, Cuesta A, Meseguer J, Balebona M C, Moriñigo M T, Esteban M T (2015) Modulation of immunity and gut microbiota after dietary administration of alginate encapsulated Shewanella putrefaciens Pdp11 to gilthead seabream (Sparus aurata L.). Fish and Shellfish Immunology, 45, 608-618.

Duran, G. M., Marshall, D. L., 2005. Ready-to-eat shrimp as an international vehicle of antibiotic-resistant bacteria. Journal of food protection. 68, 2395-2401.

Ellis A E (1990) Lysozyme assays. In: Techniques in Fish Immunology (ed. by Stolen J S, Fletcher T C, Anderson B S, Van Muiswinkel W B), SOS Publications, New Jersey, 101-103.

Kulshreshtha G, Rathgeber B, MacIsaac J, Boulianne M, Brigitte L, Stratton G, Thomas N A, Critchley A T, Hafting J, Prithiviraj B (2017) Feed Supplementation with Red Seaweeds, Chondrus crispus and Sarcodiotheca gaudichaudii, Reduce Salmonella Enteritidis in Laying Hens. Frontiers in Microbiology, 8.

Langer, Science (1990) 249, 1527

Montero D, Tort L, Izquierdo M S, Robaina L, Vergara J M (1998) Depletion of serum alternative complement pathway activity in gilthead seabream caused by α-tocopherol and n-3 HUFA dietary deficiencies. Fish Physiology and Biochemistry, 18 (4), 399-407.

Nawaz, A., Bakhsh javaid, A., Irshad, S., Hoseinifar, S. H., Xiong, H., 2018. The functionality of prebiotics as immunostimulant: Evidences from trials on terrestrial and aquatic animals. Fish & Shellfish Immunology. 76, 272-278.

O'Doherty J V, Bouwhuis M A, Sweeney T (2017) Novel marine polysaccharides and maternal nutrition to stimulate gut health and performance in post-weaned pigs. Animal Production Science, 57, 2376-2385.

Pahor-Filho, E., Júnior, J. P., Pilarski, F., Urbinati, E. C., 2017. Levamisole reduces parasitic infection in juvenile pacu (Piaractus mesopotamicus). Aquaculture. 470, 123-128.

Paul N A, de Nys R, Steinberg P D (2006) Chemical defense against bacteria in the red alga Asparagopsis armata: linking structure with function. Marine Ecology Progress Series, 306, 87-101.

Pionnier N, Falco A, Miest J, Frost P, Irnazarow I, Shrive A, Hoole D (2013) Dietary β-glucan stimulate complement and C-reactive protein acute phase responses in common carp (Cyprinus carpio) during an Aeromonas salmonicida infection. Fish & Shellfish Immunology, 34, 819-831.

Plouffe D A, Hanington P C, Walsh J G, Wilson E C, Belosevic M (2005) Comparison of select innate immune mechanisms of fish and mammals. Xenotransplantation, 12, 266-277.

Remington The Science and Practice of Pharmacy, 19th ed., Mack Printing Company, Easton, Pa. (1995)

Riera Romo M, Pérez-Martinez D, Castillo Ferrer C (2016) Innate immunity in vertebrates: an overview. Immunology, 148, 125-139.

Romero, J., Feijo\dot{o}, C. G., Navarrete, P., 2012. Antibiotics in aquaculture—use, abuse and alternatives, Health and environment in aquaculture. InTech.

Roque B M, Brooke C G, Ladau J, Polley T, Marsh L, Najafi N, Pandey P, Singh L, Salwen J K, Eloe-Fadrosh E, Kebreab E, Hess M (2018) Effect of the macroalgae Asparagopsis taxiformis on methane production and the rumen microbiome assemblage. bioRxiv.

Secombes, C J (1990). Isolation of salmonid macrophages and analysis of their killing activity. Techniques in fish immunology. 137-154.

Sohn, K. S., Kim, M. K., Kim, J. D., Han, I. K., 2000. The Role of Immunostimulants in Monogastric Animal and Fish - Review. Asian-Australas J Anim Sci. 13, 1178-1187.

Stasiak S A, Baumann P C (1996) Neutrophil activity as a potential bioindicator for contaminant analysis, Fish & Shellfish Immunology, 6, 537-539.

Sunyer O J, Tort L (1995) Natural hemolytic and bactericidal activities of sea bream Sparus aurata serum are effected by the alternative complement pathway. Veterinary Immunology and Immunopathology, 45, 333-345.

Vallejos-Vidal, E., Reyes-López, F., Teles, M., MacKenzie, S., 2016. The response of fish to immunostimulant diets. Fish and Shellfish Immunology. 56, 34-69.

Van Doan H, Doolgindachbaporn 5, Suksri A (2014) Effects of low molecular weight agar and Lactobacillus plantarum on growth performance, immunity, and disease resistance of basa fish (Pangasius bocourti, Sauvage 1880). Fish & Shellfish Immunology, 41, 340-345.

Verges A, Paul N A, Steinberg P D (2008) Sex and life-history stage alter herbivore responses to a chemically defended red alga. Ecology 89, 1334-1343.

Vidal S, Tapia-Paniagua S T, Moriñigo J M, Lobo C, Garda de la Banda I, Balebona MDC, Moriñigo MÁ (2016) Effects on intestinal microbiota and immune genes of Solea senegalensis after suspension of the administration of Shewanella putrefaciens Pdp11. Fish and Shellfish Immunology, 58, 274-283.

Wang E, Wang J, Long B, Wang K, He Y, Yang Q, Chen D, Gang Y, Huang X, Ouyang P, Lai W (2016) Molecular cloning, expression and the adjuvant effects of interleukin-8 of channel catfish (Ictalurus Punctatus) against Streptococcus iniae. Scientific Reports, 6, 29310.

1. A method for increasing the haemolytic activity of the alternative complement pathway in fish, comprising the step of administering to a fish an effective amount of a red seaweed of Asparagopsis species, an extract therefrom or residual biomass following an extraction process.

2. The method of claim 1, wherein the red seaweed is Asparagopsis taxiformis.

3. The method of claim 1, wherein the red seaweed, an extract therefrom or residual biomass following an extraction process, is administered in powdered form.

4. The method of claim 3, wherein the red seaweed is reduced to a particle size of from 10 to 1000 microns.

5. The method of claim 1, wherein an extract of the red seaweed with a solvent is administered.

6-9. (canceled)

10. The method of claim 1, wherein biomass remaining following extraction of the red seaweed with a solvent is administered.

11. The method of 1, wherein administration of the red seaweed, an extract therefrom or residual biomass following an extraction process, is by feeding the fish an animal feed containing the red seaweed.

12-23. (canceled)

24. A method for increasing phagocytic activity in fish, comprising the step of administering to a fish an effective amount of a red seaweed of Asparagopsis species, an extract therefrom or residual biomass following an extraction process.

25. The method of claim 24, wherein the red seaweed is Asparagopsis taxiformis.

26. The method of claim 24, wherein the red seaweed, an extract therefrom or residual biomass following an extraction process, is administered in powdered form.

27. The method of claim 26, wherein the red seaweed is reduced to a particle size of from 10 to 1000 microns.

28. The method of claim 24, wherein an extract of the red seaweed with a solvent is administered.

29-32. (canceled)

33. The method of claim 24, wherein biomass remaining following extraction of the red seaweed with a solvent is administered.

34. The method of claim 24, wherein administration of the red seaweed, an extract therefrom or residual biomass following an extraction process, is by feeding the fish an animal feed containing the red seaweed.

35-46. (canceled)

47. A method for boosting innate immunity in fish, comprising administering to a fish an effective amount of a red seaweed of Asparagopsis species, an extract therefrom or residual biomass following an extraction process, to activate the alternative complement pathway and increase phagocytic activity and one or more of a further seaweed selected from the group consisting of Sarconema sp., Gracilaria sp., Kappaphycus sp., Sargassum sp., Dictyota sp., Lobophora sp., Halimeda sp., Caulerpa sp., Ulva sp., a cyanobacteria of Arthrospira/Spirulina sp. and the microalga Haematococcus pluvialis, or an extract therefrom or the residue of the further seaweed following an extraction process, to increase respiratory burst activity and/or lysozyme activity.

48. The method of claim 47, wherein the red seaweed is Asparagopsis taxiformis.

49. The method of claim 47, wherein the red seaweed, an extract therefrom or residual biomass following an extraction process, is administered in powdered form.

50. The method of claim 49, wherein the red seaweed is reduced to a particle size of from 10 to 1000 microns.

51. The method of claim 47, wherein an extract of the red seaweed with a solvent is administered.

52-70. (canceled)
